(19) United States
(12) Patent Application Publication
(10) Pub. No.: US 2010/0160363 A1
(43) Pub. Date: Jun. 24, 2010

(54) EXTENDED-RELEASE PHARMACEUTICAL FORMULATIONS

(75) Inventors: John R. Cardinal, Wilmington, NC (US); Jack Lawrence James, Castle Hayne, NC (US); Elsie Melsopp, Wilmington, NC (US); David M. Oakley, Holly Springs, NC (US)

Correspondence Address: WARD AND SMITH, P.A., 1001 COLLEGE COURT, P.O. BOX 867, NEW BERN, NC, 28563-0867, US

(73) Assignee: AAIPHARMA SERVICES CORP., Wilmington, NC (US)

(21) Appl. No.: 12/641,588

(22) Filed: Dec. 18, 2009

Related U.S. Application Data
(63) Continuation-in-part of application No. 12/339,529, filed on Dec. 19, 2008.

(51) Int. Cl. A61K 31/485; A61K 31/135; A61K 31/44; A61K 31/195; A61K 31/16

(52) U.S. Cl. 514/282; 514/653; 514/355; 514/567; 514/356; 514/625

(57) ABSTRACT

The present invention provides matrix-forming, sustained-release pharmaceutical formulations comprising four primary components: i) an effective amount of at least one drug substance; ii) at least one pharmaceutically acceptable, water-swellable, pH independent polymer; iii) at least one pharmaceutically-acceptable, anionic, pH dependent polymer; and (iv) a pharmaceutically-acceptable polymer selected from the group consisting of a) at least one pharmaceutically-acceptable cationic polymer; and b) at least one pharmaceutically acceptable hydrocolloid. The present formulations can be used with compounds having a wide range of solubilities as well as compounds characterized as having hydrophobic or hydrophilic characteristics.

### RELATED APPLICATIONS

This patent application is a continuation-in-part of U.S. patent application Ser. No. 12/339,529, filed on Dec. 19, 2008, entitled “Extended-Release Pharmaceutical Formulations,” the entire disclosure of which is incorporated herein by reference.

### FIELD OF THE INVENTION

The present invention relates to controlled-release pharmaceutical compositions.

### BACKGROUND OF THE INVENTION

An objective of drug development continues to be the achievement of the delivery of optimal drug therapy. The disease states to be treated, the timing of drug release and the chemical and physical characteristics of a drug substance, among other factors, can influence the degree of success of obtaining optimal therapy. The use of controlled release, also known as extended release, drug products can deliver the desired drug therapy, with an acceptable therapeutic index (drug safety and efficacy), over an extended period of time lasting from about four hours up to about twenty-four hours. Controlled release formulations reduce the frequency of dosing for enhanced patient compliance, and can reduce the severity and frequency of side effects as they maintain desired blood levels and avoid fluctuations associated with conventional, immediate release drug products administered three to four times each day.

### SUMMARY OF THE INVENTION

The present invention provides matrix-forming, sustained-release pharmaceutical formulations comprising four primary components: i) an effective amount of at least one drug substance; ii) at least one pharmaceutically acceptable, water-swellable, pH independent polymer; at least one pharmaceutically-acceptable, anionic, pH dependent polymer; and (iv) a pharmaceutically-acceptable polymer selected from the group consisting of a) at least one pharmaceutically-acceptable cationic polymer; and b) at least one pharmaceutically acceptable hydrocolloid. These formulations are typically orally administered and have in vitro release patterns depending upon the characteristics of the surrounding environment. At gastric pH, the in vitro release pattern from these formulations is near-linear. At intestinal pH, the in vitro release pattern from these formulations is substantially a first order release pattern. Desired in vitro release patterns can be designed by manipulating the ranges and concentration of the afore-mentioned primary components. Using the compositions of the present invention, release profiles of varying time periods can be achieved using drug substances having a broad range of solubilities. To these pharmaceutical compositions can also be added pharmaceutically functional or pharmaceutically non-functional coatings. Oral dosage forms can be in the form, for example and without limitation, tablets that can be prepared by direct compression or dry or wet granulation or capsules.

### BRIEF DESCRIPTION OF THE DRAWINGS

FIG. 1: Dissolution Profile of Minocycline HCl 50 mg strength tablets from Example 1 in Varied Media.

FIG. 2: Dissolution of Delayed Release Coated 270 mg strength tablets 1-methylnicotinamide chloride Tablets from Example 2 in pH 6.8 Phosphate Buffer.

FIG. 3: Comparison of Diclofenac Potassium 50 mg strength tablets from Example 4 Prepared by Direct Compression vs. Top Spray Wet Granulation.

FIG. 4: Dissolution of Nifedipine 50 mg strength tablets vs. minitabs from Example 5 in 0.1N HCL with 1% SLS.

FIG. 5: Dissolution Profile of Diclofenac 50 mg strength tablets from Example 6 in pH buffer 6.8.

FIG. 6: Comparison of 50 mg Strength Diclofenac Potassium strength tablets from Example 6 Prepared by Direct Compression versus High Shear Wet Granulation.

FIG. 7: Dissolution of Acetaminophen 50 mg strength tablets as Prepared in Examples 7 and 8 in varied media.

FIG. 8: Dissolution of Acetaminophen 50 mg strength tablets as prepared in Examples 7 and 8 in pH buffer 6.8.

FIG. 9: Dissolution of Acetaminophen 50 mg strength tablets as prepared in Example 9.

FIG. 10: Dissolution of Nifedipine 50 mg strength tablets (lot 017) as Prepared in Example 10 in various media.

FIG. 11: Dissolution of Nifedipine 50 mg strength tablets (lot 020) as Prepared in Example 10 in various media.

FIG. 12: Dissolution of Nifedipine 50 mg strength tablets and Minitabs as prepared in Example 11 in 0.1N HCl medium.

FIG. 13: Dissolution of Minocycline 50 mg strength tablets as prepared in Example 12 in various media.

FIG. 14: Dissolution of 1-methylnicotinamide chloride 270 mg strength tablets using hypromellose vs. HPS vs. ethyl cellulose as prepared in Example 13 in pH 6.8 buffer.

FIG. 15: Dissolution of fast-release morphine sulfate 15 and 30 mg strength tablets using the preparations from Examples 16-17.

FIG. 16: Dissolution of slow-release v. fast-release morphine sulfate 60 mg strength tablets using the preparations from Examples 20-21.

FIG. 17: Dissolution of fast-release morphine sulfate 100 mg strength tablets from Example 19.

FIG. 18: Dissolution of fast-release morphine sulfate 200 mg strength tablets from Example 20.

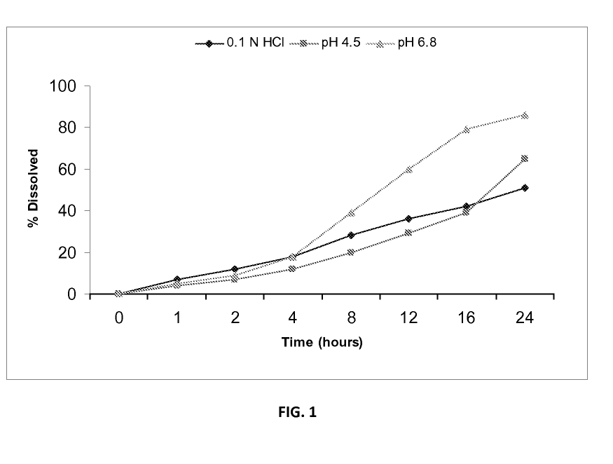

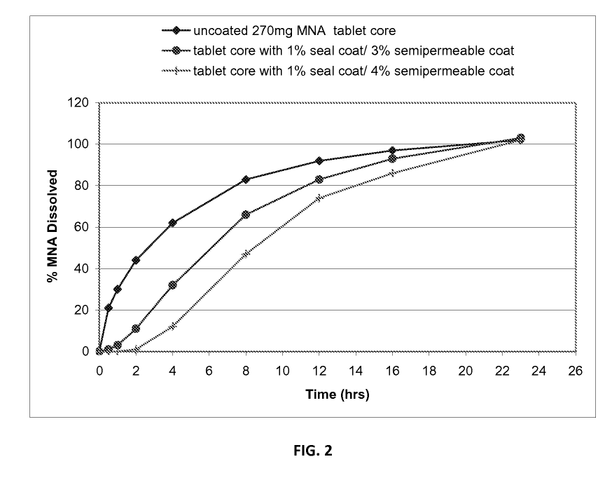

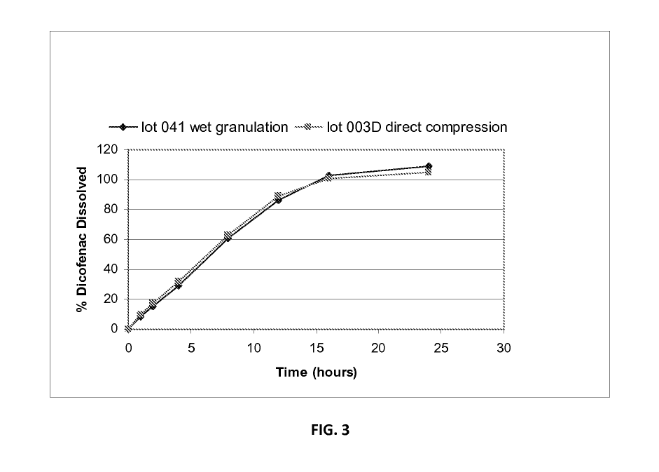

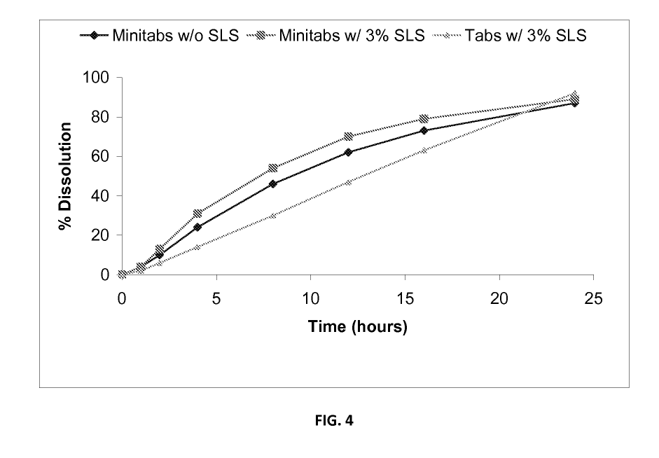

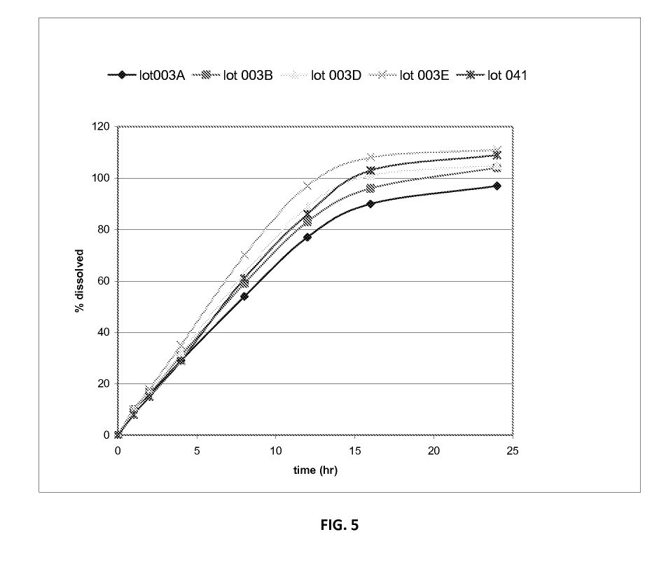

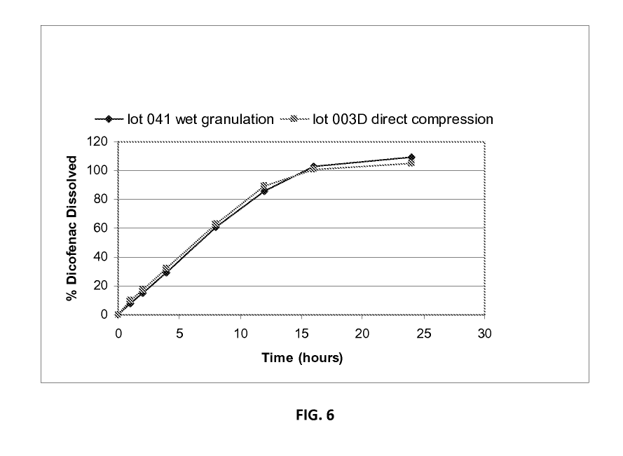

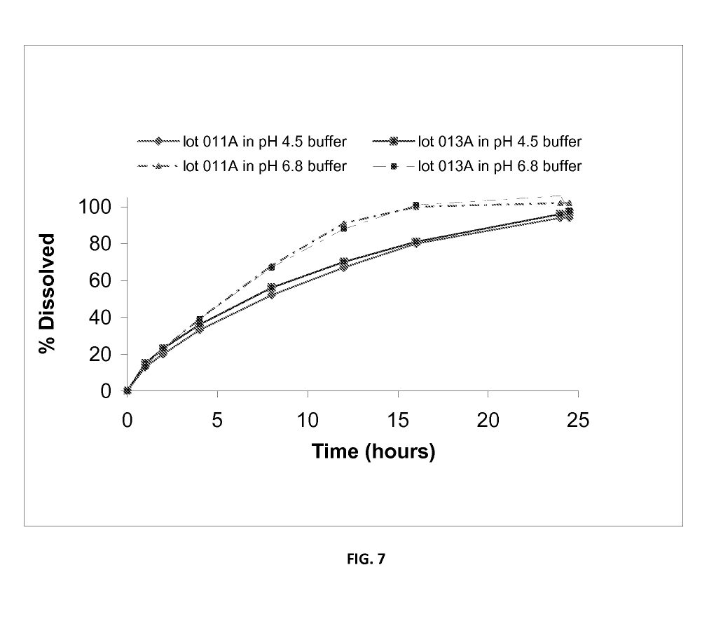

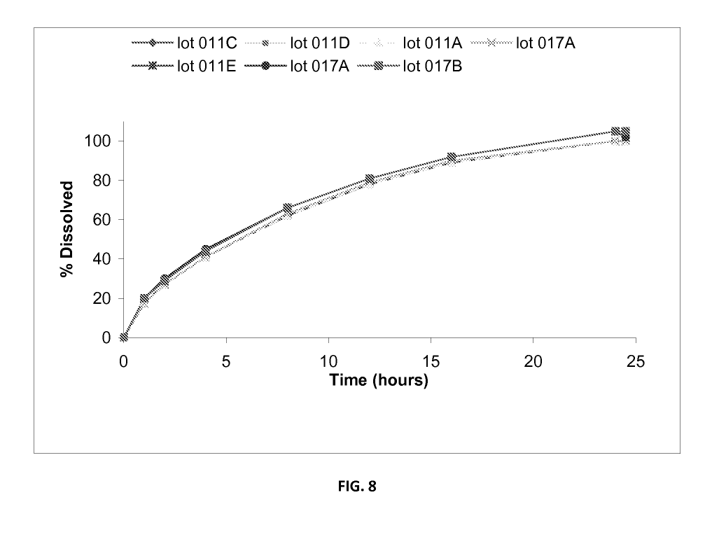

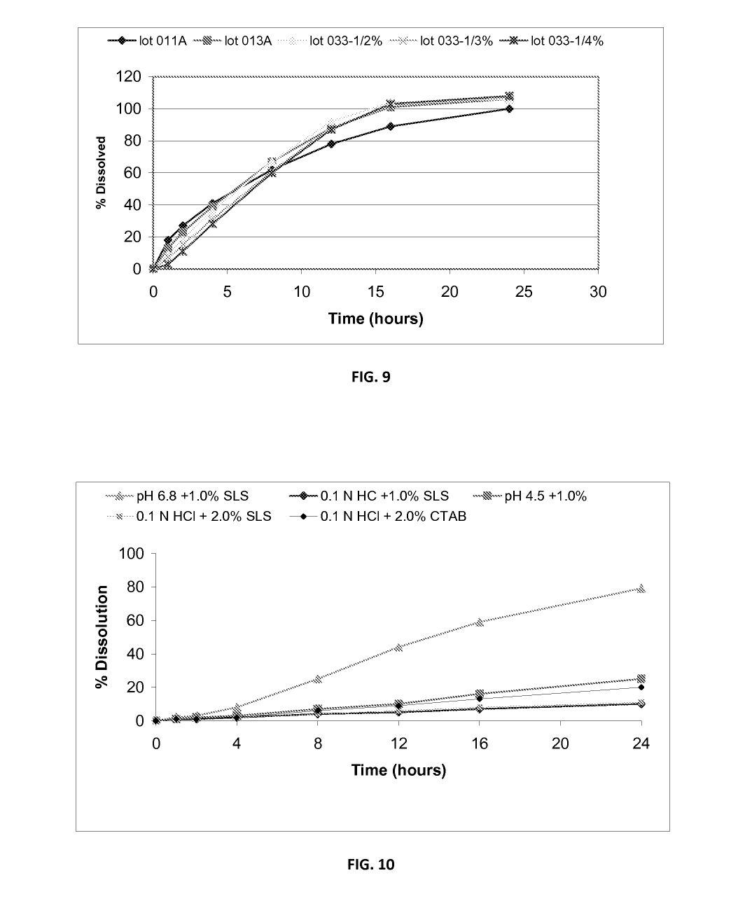

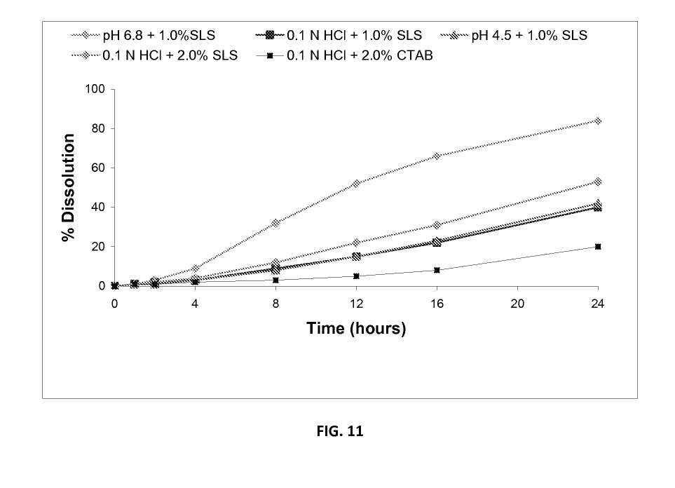

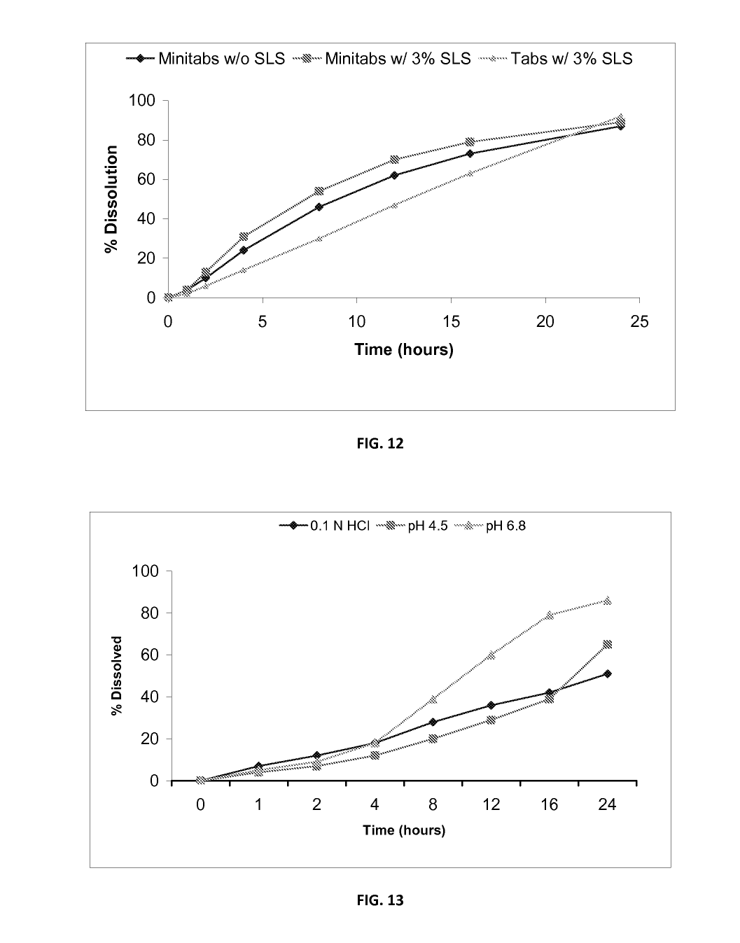

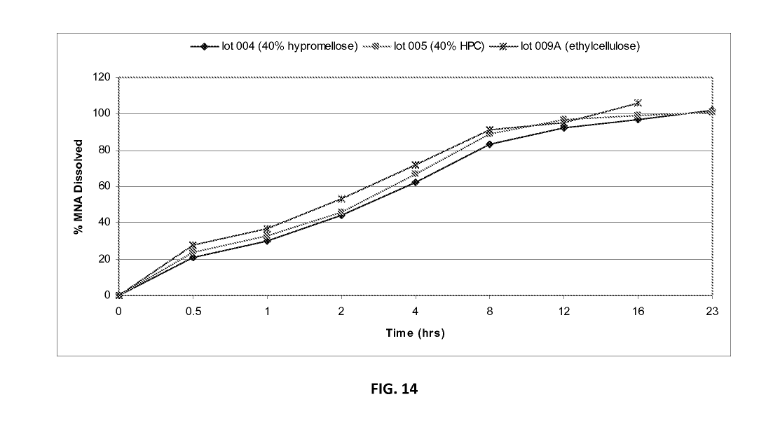

### DETAILED DESCRIPTION OF THE INVENTION

It is to be understood that unless otherwise indicated, this invention is not limited to specific active agents, vehicles, excipients, dosage forms, or the like, as such may vary. It is also to be understood that the terminology used herein is for the purpose of describing particular embodiments only, and is not intended to be limiting.

As used in this specification and the appended claims, the singular forms “a,” “an” and “the” include plural referents unless the context clearly dictates otherwise. Thus, for example, reference to “an active agent” includes a single active agent as well a two or more different active agents in combination, reference to “an excipient” includes mixtures of two or more excipients as well as a single excipient, and the like.

In describing and claiming the present invention, the following terminology will be used in accordance with the definitions set out below.

The term “delayed release” is used in its conventional sense to refer to a drug formulation in which there is a time delay provided between oral administration of a drug dosage form and the release of the drug therefrom. “Delayed release” may or may not involve gradual release of drug over an extended period of time, and thus may or may not be “sustained release.”

The terms “drug substance”, “active pharmaceutical ingredient (”API”), “pharmacologically active agent” “drug” and “agent” are used interchangeably herein to refer to any chemical compound, complex or composition that has a beneficial biological effect, generally a therapeutic effect in the treatment of a disease or abnormal physiological condition. These terms also encompass pharmaceutically acceptable, pharmacologically active derivatives of those drug substances specifically mentioned herein, including, but not limited to, salts, esters, amides, pro-drugs, active metabolites, isomers, fragments, analogs, coordination compounds and complexes, and the like. When the terms “drug substance”, active pharmaceutical ingredient (“API”), “pharmacologically active agent” “drug” and “agent” are used, then, or when a particular active agent is specifically identified, it is to be understood that applicants intend to include the active agent per se as well as pharmaceutically acceptable, pharmacologically active salts, esters, amides, pro-drugs, active metabolites, isomers, fragments, analogs, coordination compounds and complexes, and the like.

The terms “drug product” or “dosage form” denotes any form of a pharmaceutical composition that contains an amount of drug substance sufficient to achieve a therapeutic effect with a single administration. The frequency of administration that will provide the most effective results in an efficient manner without overdosing will vary with the characteristics of the particular active agent, including both its pharmacological characteristics and its physical characteristics, such as hydrophilicity.

The terms “effective amount” or a “therapeutically effective amount” of an active agent refers to a nontoxic but sufficient amount of the agent to provide the desired effect. The amount of active agent that is “effective” will vary from subject to subject, depending on the age and general condition of the individual, the particular active agent or agents, and the like. Thus, it is not always possible to specify an exact “effective amount.” However, an appropriate “effective” amount in any individual case may be determined by one of ordinary skill in the art using routine experimentation, or as recommended by an attending physician.

The term “extended release” or “sustained release” refers to a drug product in which the drug substance is gradually released over a period of time.

The term “first-order release pattern” is known by the formula F=Kt^{1/2} wherein F is the fractional release, K is a constant and t is time.

The term “gastric pH” means a pH which is less than about 4.5.

The term “immediate release” is used in its conventional sense.

The term “intestinal pH” means a pH in the range of about 5.0 to about 6.8.

The term “near-linear” means, when referring to the formula set forth in the definition of “first order release pattern”, n is about zero. The term “multi-modal release pattern” refers to the release of drug substance from a drug product having at least two distinct dissolution peaks over an extended time period of at least 1 hour.

The term “aqueous solvents” refers to a liquid solution containing water.

The term “non-aqueous solvent” refers to solvents commonly used in the pharmaceutical arts that are organic or inorganic in nature and do not contain water.

By “pharmaceutically acceptable,” such as in the recitation of a “pharmaceutically acceptable excipient,” or a “pharmaceutically acceptable additive,” is meant a material that is not biologically or otherwise undesirable, i.e., the material may be incorporated into a pharmaceutical composition administered to a patient without causing any undesirable biological effects or interacting in a deleterious manner with any of the other components of the composition in which it is contained.

The term “pharmaceutically-functional coating” refers to one or more coatings as known in the pharmaceutical arts that can influence, contribute to or inhibit the release of drug substance upon administration and, include, for example and without limitation, enteric coatings for the delayed-release of a drug substance; or, for example and without limitation, coatings that contain one or more drug substance to provide multiple phases of drug release and wherein such drug substance in a coating may be the same or different drug substance that is contained in the remainder of the dosage form.

The term “pharmaceutically non-functional coating” refers to one or more coating as known in the pharmaceutical arts that does not influence, contribute to or inhibit the release of drug substance upon administration.

The term “polymer” as used herein refers to a molecule containing a plurality of covalently attached monomer units, and includes branched, dendrimeric and star polymers as well as linear polymers. The term also includes both homopolymers and copolymers, e.g., random copolymers, block copolymers and graft copolymers, as well as uncrosslinked polymers and slightly to moderately to substantially crosslinked polymers.

The terms “treating” and “treatment” as used herein refer to reduction in severity and/or frequency of symptoms, elimination of symptoms and/or underlying cause, prevention of the occurrence of symptoms and/or their underlying cause, and improvement or remediation of damage. Thus, for example, “treating” a patient involves prevention of a particular disorder or adverse physiological event in a susceptible individual as well as treatment of a clinically symptomatic individual by inhibiting or causing regression of a disorder or disease.

The term “zero-order release pattern” can be described by the formula F α Kt and refers to a characterization of the release of a drug substance from a drug product in which at least a portion of the release pattern in graph form of the fraction of drug substance released versus time is near linear.

The present invention provides:

A matrix-forming, sustained-release pharmaceutical formulation comprising:
i) an effective amount of at least one drug substance;
ii) at least one water-swellable, pH independent polymer;
iii) at least one anionic, pH-dependent, gel-forming copolymer; and
iv) at least one polymer selected from the group consisting of:
   a. a cationic polymer; and
   b. a hydrocolloid.

The present pharmaceutical formulations can be designed for oral or other routes of administration and can be prepared such that the final drug product is substantially free of non-aqueous solvent.

The drug substances that may be administered using the pharmaceutical formulations of the present invention are not limited, as the invention enables the effective delivery of a wide variety of drug substances. Therefore, the drug substance(s) administered may be selected from any of the various classes of such drug substances including, but not limited to, analgesic agents, anesthetic agents, anti-anginal agents, antiarthritic agents, anti-arrhythmic agents, antiasthmatic agents, antibacterial agents, anti-BPH agents, anticancer agents, anticholinergic agents, anticoagulants, anticonvulsants, antidepressants, antidiabetic agents, antidiarrheals, anti-epileptic agents, antifungal agents, antigout agents, antihelminthic agents, antihistamines, antihypertensive agents, antiinflammatory agents, antimalarial agents, antimigraine agents, antimuscarinic agents, antinauseants, antineoplastic agents, anti-obesity agents, antiosteoporosis agents, antiparkinsonism agents, antiprotozoal agents, antipruritics, antipsychotic agents, antipyretics, antispasmodics, antithyroid agents, antitubercular agents, antiulcer agents, anti-urinary incontinence agents, antiviral agents, anxiolytics, appetite suppressants, attention deficit disorder (ADD) and attention deficit hyperactivity disorder (ADHD) drugs, calcium channel blockers, cardiac inotropic agents, beta-blockers, central nervous system stimulants, cognition enhancers, corticosteroids, COX-2 inhibitors, decongestants, diuretics, gastrointestinal agents, genetic materials, histamine receptor antagonists, hormonolytics, hypnotics, hypoglycemic agents, immunosuppressants, keratolytics, leukotriene inhibitors, lipid-regulating agents, macrolides, mitotic inhibitors, muscle relaxants, narcotic antagonists, nutraceiticals, neuroleptic agents, nicotine, nutritional oils, parasympatholytic agents, sedatives, sex hormones, sympathomimetic agents, tranquilizers, vasodilators, vitamins, and combinations thereof. Some agents, as will be appreciated by those of ordinary skill in the art, and as may be deduced from the discussion below, are encompassed by two or more of the aforementioned groups.

The drug substance can be hydrophobic, amphiphilic, or hydrophilic. The intrinsic water solubility of those drug substances referred to as “hydrophobic” herein, i.e., the aqueous solubility of the drug substances in electronically neutral, non-ionized form, is generally less than 1% by weight, and typically less than 0.1% or 0.01% by weight. Hydrophilic and amphiphilic drug substances herein (which, unless otherwise indicated, are collectively referred to herein as “hydrophilic” drug substances) have apparent water solubilities of at least 0.1% by weight, and typically at least 1% by weight. Both hydrophobic drug substances and hydrophilic drug substances may be selected from any of the drug substance classes, without limitation, enumerated herein. In another method of classifying the solubility of such agents, the agent(s) selected for formulating into a formulation of the present invention may have high solubility; moderate solubility; low solubility; low to moderate solubility; or moderate to high solubility. Likewise, drug substances within these solubility classes may be selected from any of the drug substance classes, without limitation, enumerated herein. When two or more drug substances, for example, are selected for use in the present formulations, each such drug substance may be from different solubility classes.

Among the various drug substance prescription and/or over-the-counter categories referenced hereinabove, the following non-limiting examples are provided: anti-inflammatory drug substances and non-opioid analgesics including, for example and without limitation, aloxiprin, auranofin, azapropazone, azathioprine, benorylate, butorphenol, capsaicin, celecoxib, diclofenac, diflunisal, esonarimod, etodolac, fenbufen, fenoprofen calcium, flurbiprofen, ibuprofen, indomethacin, ketoprofen, ketorolac, leflunomide, meclofenamic acid, mefenamic acid, nabumetone, naproxen, novantrone, oxaprozin, oxyphenbutazone, parecoxib, phenylbutazone, piclamilast, piroxicam, rofecoxib, ropivacaine, sulindac, tetrahydrocannabinol, tramadol, tromethamine, valdecoxib, and ziconotide, as well as the urinary analgesics phenazopyridine and tolterodine;

anti-angina drug substances including, for example and without limitation, mibefradil, refludan, nahnefene, carvedilol, cromafiban, lamifiban, fasudil, ranolazine, tedisamil, nisoldipine, and tizanidine;

antihelminthics including, for example and without limitation, albendazole, bephenium hydroxynaphthoate, cambendazole, dichlorophen, ivermectin, mebendazole, oxamniquine, oxfendazole, oxantel embonate, praziquantel, pyrantel embonate and thiabendazole;

anti-arrhythmic agents, such as amiodarone, disopyramide, flecainide acetate and quinidine sulfate;

anti-asthma drug substances including, for example and without limitation, zileuton, zafirlukast, terbutaline sulfate, montelukast, and albuterol;

anti-bacterial drug substances including, for example and without limitation, alatrofloxacin, azithromycin, baclofen, benethamine penicillin, cinoxacin, ciprofloxacin, clarithromycin, clofazimine, cloxacillin, demeclocycline, dirithromycin, doxycycline, erythromycin, ethionamide, furazolidone, grepafloxacin, imipenem, levofloxacin, lorefloxacin, moxifloxacin, nalidixic acid, nitrofurantoin, norfloxacin, ofloxacin, rifampicin, rifabutine, rifapentine, sparfloxacin, spiramycin, sulphabenzamide, sulphadoxine, sulphamerazine, sulphacetamide, sulphadiazine, sulphafurazole, sulphamethoxazole, sulphapyridine, tetracycline, trimethoprim, trovafloxacin, and vancomycin;

anti-cancer drug substances and immunosuppressants including, for example and without limitation, alitretinoin, aminoglutethimide, amsacrine, anastrozole, azathioprine, bexarotene, bicalutamide, biricodar, bisantrene, busulfan, camptothecin, candoxatril, capecitabine, cytarabine, chlorambucil, cyclosporin, dacarbazine, decitabine, ellipticine, estramustine, etoposide, gemcitabine, irinotecan, lasofoxifene, letrozole, lomustine, melphalan, mercaptopurine, methotrexate, mitomycin, mitotane, mitoxantrone, mofetil, mycophenolate, nebivolol, nilutamide, paclitaxel, palonosetron, procarbazine, ramipril, rubitecan, sirolimus, tacrolimus, tamoxifen, teniposide, testolactone, thalidomide, tirapazamine, topotecan, toremifene citrate, vitamin A, vitamin A derivatives, and zacopride;

anti-coagulants and other drug substancess for preventing and treating stroke including, for example and without limitation, cilostazol, citicoline, clopidogrel, cromafiban, dexanabinol, dicumarol, dipyridamole, nicoumalone, oprelvekin, perindopril erbumine, phenindione, ramipril, repinotan, ticlopidine, tirofiban, and heparin, including heparin salts formed with organic or inorganic bases, and low molecular weight heparin, i.e., heparin fragments generally having a weight average molecular weight in the range of about 1000 to about 10,000 D and exemplified by enoxaparin, dalteparin, danaproid, gammaparin, nadroparin, ardeparin, tinzaparin, certoparin, and reviparin;

anti-diabetic drug substances include, for example and without limitation, acetohexamide, chlorpropamide, ciglitazone, farglitazar, glibenclamide, gliclazide, glipizide, glucagon, glyburide, glymepiride, miglitol, nateglinide, pimagedine, pioglitazone, repaglinide, rosiglitazone, tolazamide, tolbutamide, triampterine, troglitazone and voglibose;

anti-epileptics including, for example and without limitation, beclamide, carbamazepine, clonazepam, ethotoin, felbamate, fosphenytoin, lamotrigine, methoin, methsuximide, methylphenobarbitone, oxcarbazepine, paramethadione, phenacemide, phenobarbitone, phenytoin, phensuximide, primidone, sulthiame, tiagabine, topiramate, valproic acid, and viga batrin;

anti-fungal drug substances including, for example and without limitation, amphotericin, butenafine, butoconazole nitrate, clotrimazole, econazole nitrate, fluconazole, flucytosine, griseofulvin, itraconazole, ketoconazole, miconazole, natamycin, nystatin, sulconazole nitrate, oxiconazole, terbinafine, terconazole, tioconazole and undecenoic acid;

anti-gout drug substances including, for example and without limitation, allopurinol, probenecid and sulphin-pyrazone;

antihistamines and allergy medications including, for example and without limitation, acrivastine, astemizole, chlorpheniramine, cinnarizine, cetirizine, clemastine, cyclizine, cyproheptadine, desloratadine, dexchlorpheniramine, dimenhydrinate, diphenhydramine, epinastine, fexofenadine, flunarizine, loratadine, meclizine, mizolastine, oxatomide, and terfenadine;

antihypertensive drug substances include, for example and without limitation, amlodipine, benazepril, benidipine, candesartan, captopril, carvedilol, darodipine, dilitazem, diazoxide, doxazosin, enalapril, epleronone, eposartan, felodipine, fenoldopam, fosinopril, guanabenz, iloprost, irbesartan, isradipine, lercardinipine, lisinopril, losartan, minoxidil, nebivolol, nicardipine, nifedipine, nimodipine, nisoldipine, omapatrilat, phenoxybenzamine, prazosin, quinapril, reserpine, semotiadil, sitaxsentan, terazosin, telmisartan, and valsartan.

anti-malarials including, for example and without limitation, amodiaquine, chloroquine, chlorproguanil, halofantrine, mefloquine, proguanil, pyrimethamine and quinine sulfate;

drug substances for treating headaches, including anti-migraine agents including, for example and without limitation, almotriptan, butorphanol, dihydroergotamine, dihydroergotamine mesylate, eletriptan, ergotamine, frovatriptan, methysergide, naratriptan, pizotyline, rizatriptan, sumatriptan, tonaberstat, and zolmitriptan;

anti-muscarinic drug substances including, for example and without limitation, atropine, benzhexol, biperiden, ethopropazine, hyoscyamine, mepenzolate bromide, oxyphencyclimine, scopolamine, and tropicamide;

anti-protozoal drug substances including, for example and without limitation, atovaquone, benznidazole, clioquinol, decoquinate, diiodohydroxyquinoline, diloxanide furoate, dinitolmide, furazolidone, metronidazole, nimorazole, nitrofirazone, ornidazole and tinidazole;

anti-thyroid drug substances including, for example and without limitation, carbimazole, paricalcitol, and propylthiouracil;

anti-tussives including, for example and without limitation, benzonatate;

antiviral drug substances include, for example and without limitation, antiherpes agents acyclovir, famciclovir, foscarnet, ganciclovir, idoxuridine, sorivudine, trifluridine, valacyclovir, and vidarabine, and otherantiviral agents such as abacavir, amantadine, amprenavir, delviridine, didanosine, efavirenz, indinavir, interferon alpha, lamivudine, nelfinavir, nevirapine, ribavirin, rimantadine, ritonavir, saquinavir, stavudine, tipranavir, valganciclovir, zalcitabine, and zidovudine; and other antiviral agents such as abacavir, indinavir, interferon alpha, nelfinavir, ribavirin, rimantadine, tipranavir, ursodeoxycholic acid, and valganciclovir.

anxiolytics, sedatives, and hypnotics including, for example and without limitation, alprazolam, amylobarbitone, barbitone, bentazepam, bromazepam, bromperidol, brotizolam, butobarbitone, carbromal, chlordiazepoxide, chlormethiazole, chlorpromazine, chlorprothixene, clonazepam, clobazam, clotiazepam, clozapine, dexmethylphenidate (d-threo-methylphenidate) diazepam, droperidol, ethinamate, flunanisone, flunitrazepam, triflupromazine, flupenthixol decanoate, fluphenazine, flurazepam, gabapentin, gaboxadol, .gamma.-hydroxybutyrate, haloperidol, lamotrigine, lorazepam, lormetazepam, medazepam, meprobamate, mesoridazine, methaqualone, methylphenidate, midazolam, modafinil, molindone, nitrazepam, olanzapine, oxazepam, pentobarbitone, perphenazine pimozide, pregabalin, prochlorperazine, pseudoephedrine, quetiapine, rispiridone, sertindole, siramesine, sulpiride, sunepitron, temazepam, thioridazine, triazolam, zaleplon, zolpidem, and zopiclone;

appetite suppressants, anti-obesity drug substances and drug substances for treatment of eating disorders including, for example and without limitation, amphetamine, bromocriptine, dextroamphetamine, diethylpropion, lintitript, mazindol, methamphetamine, orlistat, phentermine, and topiramate;

cardiovascular drug substances including, for example and without limitation, angiotensin converting enzyme (ACE) inhibitors such as enalapril, ramipril, perindopril erbumine, 1-carboxymethyl-3-1-carboxy-3-phenyl-(1S)-propylamino-2,3,4,5-tetrahydro-1H-(3S)-1-benzazepine-2-one, 3-(5-amino-1-carboxy-1S-pentyl)amino-2,3,4,5-tetrahydro-2-oxo-3S-1H-1-ben-zazepine-1acetic acid or 3-(1-ethoxycarbonyl-3-phenyl-(1S)-propylamino)-2,3,4,5-tetrahydro-2-oxo-(-3S)-benzazepi acid monohydrochloride; cardiac glycosides and cardiac inotropes such as amrinone, digoxin, digitoxin, enoximone, lanatoside C, medigoxin, and milrinone; calcium channel blockers such as verapamil, nifedipine, nicardipene, felodipine, isradipine, nimodipine, amlodipine and diltiazem; beta-blockers such as acebutolol, alprenolol, atenolol, labetalol, metoprolol, nadolol, oxyprenolol, pindolol, propafenone, propranolol, esmolol, sotalol, timolol, and acebutolol; antiarrhythmics such as moricizine, dofetilide, ibutilide, nesiritide, procainamide, quinidine, disopyramide, lidocaine, phenytoin, tocainide, mexiletine, flecainide, encainide, bretylium and amiodarone; cardioprotective agents such as dexrazoxane and leucovorin; vasodilators such as nitroglycerin; diuretic agents such as azetazolamide, amiloride, bendroflumethiazide, bumetanide, chlorothiazide, chlorthalidone, ethacrynic acid, furosemide, hydrochlorothiazide, metolazone, nesiritide, spironolactone, and triamterine; and miscellaneous cardiovascular drugs such as monteplase and corlopam;

corticosteroids including, for example and without limitation, beclomethasone, betamethasone, budesonide, cortisone, desoxymethasone, dexamethasone, fludrocortisone, flunisolide, fluocortolone, fluticasone propionate, hydrocortisone, methylprednisolone, prednisolone, prednisone and triamcinolone;

erectile dysfunction drug substances including, for example and without limitation, pomorphine, phentolamine, and vardenafil;

gastrointestinal drug substances including, for example and without limitation, alosetron, bisacodyl, cilansetron, cimetidine, cisapride, diphenoxylate, domperidone, esomeprazole, famotidine, granisetron, lansoprazole, loperamide, mesalazine, nizatidine, omeprazole, ondansetron, prantoprazole, rabeprazole sodium, ranitidine, risperidone, sulphasalazine, and tegaserod;

genetic material including, for example and without limitation, nucleic acids, RNA, DNA, recombinant RNA, recombinant DNA, antisense RNA, antisense DNA, ribozymes, ribooligonucleotides, deoxyribonucleotides, antisense ribooligonucleotides, and antisense deoxyribooligonucleotides. Representative genes include those encoding for vascular endothelial growth factor, fibroblast growth factor, Bcl-2, cystic fibrosis transmembrane regulator, nerve growth factor, human growth factor, erythropoietin, tumor necrosis factor, and interleukin-2, as well as histocompatibility genes such as HLA-B7.

keratolytics including, for example and without limitation, acetretin, calcipotriene, calcifediol, calcitriol, cholecalciferol, ergocalciferol, etretinate, retinoids, targretin, and tazarotene;

Lipid-regulating drug substances that are generally classified as hydrophobic include HMG CoA reductase inhibitors including, for example and without limitation, atorvastatin, simvastatin, fluvastatin, pravastatin, lovastatin, cerivastatin, rosuvastatin, and pitavastatin, as well as other lipid-lowering (“antihyperlipidemic”) drug substances such as 1-methylnicotinamide chloride (1-MNA) HCl, bezafibrate, beclobrate, binifibrate, ciprofibrate, clinofibrate, clofibrate, clofibric acid, ezetimibe, etofibrate, fenofibrate, fenofibric acid, gemfibrozil, niacin, nicofibrate, pirifibrate, probucol, ronifibrate, simfibrate, and theofibrate.

muscle relaxants including, for example and without limitation, cyclobenzaprine, dantrolene sodium and tizanidine HCl;

agents to treat neurodegenerative diseases, including active drug substances for treating Alzheimer's disease including, for example and without limitation, akatinol, donezepil, donepezil hydrochloride, dronabinol, galantamine, neotrofin, rasagiline, physostigmine, physostigmine salicylate, propentoffyline, quetiapine, rivastigmine, tacrine, tacrine hydrochloride, thalidomide, and xaliproden; drug substances for treating Huntington's Disease including, for example and without limitation, fluoxetine and carbamazepine; anti-parkinsonism drugs useful such as, without limitation amantadine, apomorphine, bromocriptine, entacapone, levodopa (particularly a levodopa/carbidopa combination), lysuride, pergolide, pramipexole, rasagiline, riluzole, ropinirole, selegiline, sumanirole, tolcapone, trihexyphenidyl, and trihexyphenidyl hydrochloride; and drug substances for treating ALS such, without limitation, the anti-spastic agents baclofen, diazemine, and tizanidine;

nitrates and other anti-anginal drug substances including, for example and without limitation, amyl nitrate, glyceryl trinitrate, isosorbide dinitrate, isosorbide mononitrate and pentaerythritol tetranitrate;

neuroleptic drug substances including, for example, antidepressant drugs, antimanic drugs, and antipsychotic agents, wherein antidepressant drugs include, without limitation, (a) the tricyclic antidepressants such as amoxapine, amitriptyline, clomipramine, desipramine, doxepin, imipramine, maprotiline, nortriptyline, protriptyline, and trimipramine, (b) the serotonin reuptake inhibitors such as citalopram, fluoxetine, fluvoxamine, paroxetine, sertraline, and venlafaxine, (c) monoamine oxidase inhibitors such as phenelzine, tranylcypromine, and (−)-selegiline, and (d) other antidepressants such as aprepitant, bupropion, duloxetine, gepirone, igmesine, lamotrigine, maprotiline, mianserin, mirtazapine, nefazodone, rabalzotan, sunepitron, trazodone and venlafaxine, and wherein antimanic and antipsychotic agents include, for example and without limitation, (a) phenothiazines such as acetophenazine, acetophenazine maleate, chlorpromazine, chlorpromazine hydrochloride, fluphenazine, fluphenazine hydrochloride, fluphenazine enanthate, fluphenazine decanoate, mesoridazine, mesoridazine besylate, perphenazine, thioridazine, thioridazine hydrochloride, trifluoperazine, and trifluoperazine hydrochloride, (b) thioxanthenes such as chlorprothixene, thiothixene, and thiothixene hydrochloride, and (c) other heterocyclic drugs such as carbamazepine, clozapine, droperidol, haloperidol, haloperidol decanoate, loxapine succinate, molindone, molindone hydrochloride, olanzapine, pimozide, quetiapine, risperidone, and sertindole.

nutritional agents including, for example and without limitation, calcitriol, carotenes, dihydrotachysterol, essential fatty acids, non-essential fatty acids, phytonadiol, vitamin A, vitamin B.sub.2, vitamin D, vitamin E and vitamin K.

opioid analgesics including, for example and without limitation, alfentanil, apomorphine, buprenorphine, butorphanol, codeine, dextropropoxyphene, diamorphine, dihydrocodeine, fentanyl, hydrocodone, hydromorphone, levorphanol, meperidine, meptazinol, methadone, morphine, nalbuphine, oxycodone, oxymorphone, pentazocine, propoxyphene, sufentanil, and tramadol;

peptidyl drug substances include therapeutic peptides and proteins per se, whether naturally occurring, chemically synthesized, recombinantly produced, and/or produced by biochemical (e.g., enzymatic) fragmentation of larger molecules, and may contain the native sequence or an active fragment thereof. Specific peptidyl drugs include, for example and without limitation, the peptidyl hormones activin, amylin, angiotensin, atrial natriuretic peptide (ANP), calcitonin, calcitonin gene-related peptide, calcitonin N-terminal flanking peptide, ciliary neurotrophic factor (CNTF), corticotropin (adrenocorticotropin hormone, ACTH), corticotropin-releasing factor (CRF or CRH), epidermal growth factor (EGF), follicle-stimulating hormone (FSH), gastrin, gastrin inhibitory peptide (GIP), gastrin-releasing peptide, gonadotropin-releasing factor (GnRF or GNRH), growth hormone releasing factor (GRF, GRH), human chorionic gonadotropin (hCH), inhibin A, inhibin B, insulin, luteinizing hormone (LH), luteinizing hormone-releasing hormone (LHRH), .alpha.-melanocyte-stimulating hormone, .beta.-melanocyte-stimulating hormone, .gamma.-melanocyte-stimulating hormone, melatonin, motilin, oxytocin (pitocin), pancreatic polypeptide, parathyroid hormone (PTH), placental lactogen, prolactin (PRL), prolactin-release inhibiting factor (PIF), prolactin-releasing factor (PRF), secretin, somatotropin (growth hormone, GH), somatostatin (SIF, growth hormone-release inhibiting factor, GIF), thyrotropin (thyroid-stimulating hormone, TSH), thyrotropin-releasing factor (TRH or TRF), thyroxine, vasoactive intestinal peptide (VIP),and vasopressin. Other peptidyl drug substances are the cytokines, e.g., colony stimulating factor 4, heparin binding neurotrophic factor (HBNF), interferon-.alpha., interferon .alpha.-2a, interferon .alpha.-2b, interferon .alpha.-n3, interferon-.beta., etc., interleukin-1, interleukin-2, interleukin-3, interleukin-4, interleukin-5, interleukin-6, etc., tumor necrosis factor, tumor necrosis factor-.alpha., granuloycte colony-stimulating factor (G-CSF), granulocyte-macrophage colony-stimulating factor (GM-CSF), macrophage colony-stimulating factor, midkine (MD), and thymopoietin. Still other peptidyl drug substances include endorphins (e.g., dermorphin, dynorphin, .alpha.-endorphin, .beta.-endorphin, .gamma.-endorphin, .sigma.-endorphin, [Leu.sup.5]enkephalin, [Met.sup.5]enkephalin, substance P), kinins (e.g., bradykinin, potentiator B, bradykinin potentiator C, kallidin), LHRH analogues (e.g., buserelin, deslorelin, fertirelin, goserelin, histrelin, leuprolide, lutrelin, nafarelin, tryptorelin), and the coagulation factors, such as .alpha.sub.1-antitrypsin, .alpha.sub.2-macroglobulin, antithrombin III, factor I (fibrinogen), factor II (prothrombin), factor III (tissue prothrombin), factor V (proaccelerin), factor VII (proconvertin), factor VIII (antihemophilic globulin or AHG), factor IX (Christmas factor, plasma thromboplastin component or PTC), factor X (Stuart-Power factor), factor XI (plasma thromboplastin antecedent or PTA), factor XII (Hageman factor), heparin cofactor II, kallikrein, plasmin, plasminogen, prekallikrein, protein C, protein S, and thrombomodulin and combinations thereof. sex hormones include, for example and without limitation, progestins (progestogens), estrogens, and combinations thereof. Progestins include acetoxypregnenolone, allylestrenol, anagestone acetate, chlormadinone acetate, cyproterone, cyproterone acetate, desogestrel, dihydrogesterone, dimethisterone, ethisterone (17.alpha.-ethinyltestosterone), ethynodiol diacetate, flurogestone acetate, gestadene, hydroxyprogesterone, hydroxyprogesterone acetate, hydroxyprogesterone caproate, hydroxymethylprogesterone, hydroxymethylprogesterone acetate, 3-ketodesogestrel, levonorgestrel, lynestrenol, medrogestone, medroxyprogesterone acetate, megestrol, megestrol acetate, melengestrol acetate, norethindrone, norethindrone acetate, norethisterone, norethisterone acetate, norethynodrel, norgestimate, norgestrel, norgestrienone, normethisterone, progesterone, and trimgestone. Also included within this general class are estrogens, e.g.: estradiol (i.e., 1,3,5-estratriene-3,17.beta.-diol, or “17.beta.-estradiol”) and its esters, including estradiol benzoate, valerate, cypionate, heptanoate, decanoate, acetate and diacetate; 17.alpha.-estradiol; ethinylestradiol (i.e., 17.alpha.-ethinylestradiol) and esters and ethers thereof, including ethinylestradiol 3-acetate and ethinylestradiol 3-benzoate; estriol and estriol succinate; polyestrol phosphate; estrone and its esters and derivatives, including estrone acetate, estrone sulfate, and piperazine estrone sulfate; quinestrol; mestranol; and conjugated equine estrogens. In many contexts, e.g., in female contraception and in hormone replacement therapy (HRT), a combination of a progestin and estrogen is used, e.g., progesterone and 17.beta.-estradiol. For HRT, an androgenic agent may be advantageously included as well. Androgenic agents for this purpose include, for example, dehydroepiandrosterone (DHEA; also termed “prasterone”), sodium dehydroepiandrosterone sulfate, 4-dihydrotestosterone (DHT; also termed “stanolone”), and testosterone, and pharmaceutically acceptable esters of testosterone and 4-dihydrotestosterone, typically esters formed from the hydroxyl group present at the C-17 position, including, but not limited to, the enanthate, propionate, cypionate, phenylacetate, acetate, isobutyrate, buciclate, heptanoate, decanoate, undecanoate, caprate and isocaprate esters;

androgenic drug substances may also be administered for other purposes well known in the art. In addition to the androgenic agents enumerated above, other androgenic agents include, but are not limited to, androsterone, androsterone acetate, androsterone propionate, androsterone benzoate, androstenediol, androstenediol-3-acetate, androstenediol-17-acetate, androstenediol-3,17-diacetate, androstenediol-17-benzoate, androstenediol-3-acetate-17-benzoate, androstenedione, ethylestrenol, oxandrolone, nandrolone phenpropionate, nandrolone decanoate, nandrolone furylpropionate, nandrolone cyclohexane-propionate, nandrolone benzoate, nandrolone cyclohexanecarboxylate, stanozolol, dromostanolone, and dromostanolone propionate.

stimulants, including active drug substances for treating narcolepsy including attention deficit disorder (ADD) and attention deficit hyperactivity disorder (ADHD) including, for example and without limitation, amphetamine, dexamphetamine, dexfenfluramine, mazindol, methylphenidate (including d-threo-methylphenidate or “dexmethylphenidate”, mondafinil, pemoline and sibutramine.

Considering solubility, exemplary hydrophobic active agents include, without limitation, acetretin, acetyl coenzyme Q, albendazole, albuterol, aminoglutethimide, amiodarone, amlodipine, amphetamine, amphotericin B, atorvastatin, atovaquone, azithromycin, baclofen, beclomethasone, benazepril, benzonatate, betamethasone, bicalutanide, budesonide, bupropion, busulfan, butenafine, calcifediol, calcipotriene, calcitriol, camptothecin, candesartan, capsaicin, carbamezepine, carotenes, celecoxib, cerivastatin, cetirizine, chlorpheniramine, cholecalciferol, cilostazol, cimetidine, cinnarizine, ciprofloxacin, cisapride, clarithromycin, clemastine, clomiphene, clomipramine, clopidogrel, codeine, coenzyme Q10, cyclobenzaprine, cyclosporin, danazol, dantrolene, dexchlorpheniramine, diclofenac, dicumarol, digoxin, dehydroepiandrosterone, dihydroergotamine, dihydrotachysterol, dirithromycin, donezepil, efavirenz, eposartan, ergocalciferol, ergotamine, essential fatty acid sources, estradiol, etodolac, etoposide, famotidine, fenofibrate, fentanyl, fexofenadine, finasteride, fluconazole, flurbiprofen, fluvastatin, fosphenytoin, frovatriptan, furazolidone, gabapentin, gemfibrozil, glibenclamide, glipizide, glyburide, glimepiride, griseofulvin, halofantrine, ibuprofen, irbesartan, irinotecan, isosorbide dinitrate, isotretinoin, itraconazole, ivermectin, ketoconazole, ketorolac, lamotrigine, lansoprazole, leflunomide, lisinopril, loperamide, loratadine, lovastatin, L-thyroxine, lutein, lycopene, medroxyprogesterone, mifepristone, mefloquine, megestrol acetate, methadone, methoxsalen, metronidazole, miconazole, midazolam, miglitol, minoxidil, mitoxantrone, montelukast, nabumetone, nalbuphine, naratriptan, nelfinavir, nifedipine, nisoldipine, nilutanide, nitro furantoin, nizatidine, omeprazole, oprevelkin, oxaprozin, paclitaxel, paracalcitol, paroxetine, pentazocine, pioglitazone, pizofetin, pravastatin, prednisolone, probucol, progesterone, pseudoephedrine, pyridostigmine, rabeprazole, raloxifene, repaglinide, rifabutine, rifapentine, rimexolone, ritanovir, rizatriptan, rofecoxib, rosiglitazone, saquinavir, sertraline, sibutramine, sildenafil citrate, simvastatin, sirolimus, spironolactone, sumatriptan, tacrine, tacrolimus, tamoxifen, tamsulosin, targretin, tazarotene, telmisartan, teniposide, terbinafine, terazosin, tetrahydrocannabinol, tiagabine, ticlopidine, tirofiban, tizanidine, topiramate, topotecan, toremifene, tramadol, tretinoin, troglitazone, trovafloxacin, ubidecarenone, valsartan, venlafaxine, verteporfin, vigabatrin, vitamin A, vitamin D, vitamin E, vitamin K, zafirlukast, zileuton, zolmitriptan, zolpidem, zopiclone, and combinations thereof.

Exemplary hydrophilic active agents include, without limitation, acarbose, acyclovir, acetyl cysteine, acetylcholine chloride, alatrofloxacin, alendronate, alglucerase, amantadine hydrochloride, ambenomium, amifostine, amiloride hydrochloride, aminocaproic acid, amphotericin B, antihemophilic factor (human), antihemophilic factor (porcine), antihemophilic factor (recombinant), aprotinin, asparaginase, atenolol, atracurium besylate, atropine, azithromycin, aztreonam, BCG vaccine, bacitracin, becaplermin, belladona, bepridil hydrochloride, bleomycin sulfate, calcitonin human, calcitonin salmon, carboplatin, capecitabine, capreomycin sulfate, cefamandole nafate, cefazolin sodium, cefepime hydrochloride, cefixime, cefonicid sodium, cefoperazone, cefotetan disodium, cefotaxime, cefoxitin sodium, ceftizoxime, ceftriaxone, cefuroxime axetil, cephalexin, cephapirin sodium, cholera vaccine, chorionic gonadotropin, cidofovir, cisplatin, cladribine, clidinium bromide, clindamycin and clindamycin derivatives, ciprofloxacin, clodronate, colistimethate sodium, colistin sulfate, corticotropin, cosyntropin, cromolyn sodium, cytarabine, dalteparin sodium, danaparoid, deferoxamine, denileukin diftitox, desmopressin, diatrizoate meglumine and diatrizoate sodium, dicyclomine, didanosine, dirithromycin, dopamine hydrochloride, dornase alpha, doxacurium chloride, doxorubicin, etidronate disodium, enalaprilat, enkephalin, enoxaparin, enoxaprin sodium, ephedrine, epinephrine, epoetin alpha, erythromycin, esmolol hydrochloride, factor IX, famciclovir, fludarabine, fluoxetine, foscamet sodium, ganciclovir, granulocyte colony stimulating factor, granulocyte-macrophage stimulating factor, recombinant human growth hormone, bovine growth hormone, gentamycin, glucagon, glycopyrolate, gonadotropin releasing hormone and synthetic analogs thereof, gonadorelin, grepafloxacin, haemophilus B conjugate vaccine, hepatitis A virus vaccine inactivated, hepatitis B virus vaccine inactivated, heparin sodium, indinavir sulfate, influenza virus vaccine, interleukin-2, interleukin-3, insulin-human, insulin lispro, insulin procine, insulin NPH, insulin aspart, insulin glargine, insulin detemir, interferon alpha, interferon beta, ipratropium bromide, ifosfamide, Japanese encephalitis virus vaccine, lamivudine, leucovorin calcium, leuprolide acetate, levofloxacin, lincomycin and lincomycin derivatives, lobucavir, lomefloxacin, loracarbef, mannitol, measles virus vaccine, meningococcal vaccine, menotropins, mepenzolate bromide, mesalamine, methenamine, methotrexate, methscopolamine, metformin hydrochloride, metoprolol, mezlocillin sodium, mivacurium chloride, mumps viral vaccine, nedocromil sodium, neostigmine bromide, neostigmine methyl sulfate, neurontin, norfloxacin, octreotide acetate, ofloxacin, olpadronate, oxytocin, pamidronate disodium, pancuronium bromide, paroxetine, perfloxacin, pentamidine isethionate, pentostatin, pentoxifylline, penciclovir, pentagastrin, phentolamine mesylate, phenylalanine, physostigmine salicylate, plague vaccine, piperacillin sodium, platelet derived growth factor, pneumococcal vaccine polyvalent, poliovirus vaccine (inactivated), poliovirus vaccine live (OPV), polymyxin B sulfate, pralidoxime chloride, pramlintide, pregabalin, propafenone, propenthaline bromide, pyridostigmine bromide, rabies vaccine, risedronate, ribavirin, rimantadine hydrochloride, rotavirus vaccine, salmeterol xinafoate, sincalide, small pox vaccine, solatol, somatostatin, sparfloxacin, spectinomycin, stavudine, streptokinase, streptozocin, suxamethonium chloride, tacrine hydrochloride, terbutaline sulfate, thiopeta, ticarcillin, tiludronate, timolol, tissue type plasminogen activator, TNFR:Fc, TNK-tPA, trandolapril, trimetrexate gluconate, trospectomycin, trovafloxacin, tubocurarine chloride, tumor necrosis factor, typhoid vaccine live, urea, urokinase, vancomycin, valacyclovir, valsartan, varicella virus vaccine live, vasopressin and vasopressin derivatives, vecuronium bromide, vinblastine, vincristine, vinorelbine, vitamin B12, warfarin sodium, yellow fever vaccine, zalcitabine, zanamivir, zolendronate, zidovudine, and combinations thereof.

Of course, certain active agents indicated as hydrophobic may be readily converted to and commercially available in hydrophilic form, e.g., by ionizing a non-ionized active agent so as to form a pharmaceutically acceptable, pharmacologically active salt. Conversely, certain active agents indicated as hydrophilic may be readily converted to and commercially available in hydrophobic form, e.g., by neutralization, esterification, or the like. Thus, it should be understood that the above categorization of certain active agents as hydrophilic or hydrophobic is not intended to be limiting.

Any of the aforementioned active agents may also be administered in combination using the present formulations. Active agents administered in combination may be from the same therapeutic class (e.g., lipid-regulating agents or anticoagulants) or from different therapeutic classes (e.g., a lipid-regulating agent and an anticoagulant). Non-limiting examples of drug substance combination products include, without limitation:

female contraceptive compositions containing both a progestogen and an estrogen;

female HRT compositions containing a progestogen, an estrogen, and an androgen;

combinations of lipid-regulating agents, e.g., (a) a fibrate and a statin, such as fenofibrate and atorvastatin, fenofibrate and simvastatin, fenofibrate and lovastatin, or fenofibrate and pravastatin; (b) a fibrate and nicotinic acid, such fenofibrate and niacin; and (c) a statin and a nicotinic acid, such as lovastatin and niacin;

combinations of a lipid-regulating agent and an antiviral agent, e.g., a fibrate and a protease inhibitor, such as fenofibrate and ritonavir;

combinations of a lipid-regulating agent and an anticoagulant, e.g., (a) a fibrate and a salicylate, such as fenofibrate and aspirin, (b) a fibrate and another anticoagulant, such as fenofibrate and clopidogrel, (c) a statin and a salicylate, such as simvastatin and aspirin, and (d) a statin and another anticoagulant such as pravastatin and clopidogrel;

combinations of a lipid-regulating agent and an antidiabetic agent, including (a) a fibrate and a insulin sensitizer such as a thiazolidinedione, e.g., fenofibrate and pioglitazone, or fenofibrate and rosiglitazone, (b) a fibrate and an insulin stimulant such as a sulfonylurea, e.g., fenofibrate and glimepiride, or fenofibrate and glipizide, a statin and and insulin sensitizer such as a thiazolidinedione, e.g., lovastatin and pioglitazone, simvastatin and rosiglitazone, pravastatin and pioglitazone, or the like;

combinations of a lipid regulating agent and a cardiovascular drug, e.g., (a) a fibrate and a calcium channel blocker, such as fenofibrate and amlodipine, or fenofibrate and irbesartan, or (b) a statin and a calcium channel blocker, such as fosinopril and pravastatin;

combinations of anticoagulants, e.g., (a) a salicylate and a platelet receptor binding inhibitor, such as aspirin and clopidogrel, (b) a salicylate and a low molecular weight heparin, such as aspirin and dalteparin, and (c) a platelet receptor binding inhibitor and a low molecular weight heparin, such as clopidogrel and enoxaparin;

combinations of antidiabetics, e.g., (a) an insulin sensitizer and an insulin stimulant, such as (i) a thiazolidinedione such as glitazone or pioglitazone and a sulfonylurea such as glimepiride, and (ii) a biguanide such as metformin and a meglitinide such as repaglinide, (b) an insulin sensitizer and an .alpha.-glucosidase inhibitor, such as metformin and acarbose, (c) an insulin stimulant and an .alpha.-glucosidase inhibitor, such as (i) a sulfonylurea such as glyburide combined with acarbose, (ii) acarbose and a meglitinide such as repaglinide, (iii) miglitol and a sulfonylurea such as glipizide, (iv) acarbose and a thiazolidinedione such as pioglitazone, or (v) metformin and pioglitazone;

combinations of cardiovascular drugs, such as combinations of ACE inhibitors, e.g., lisinopril and candesartan; a combination of an ACE inhibitor with a diuretic agent such as losartan and hydrochlorothiazide; a combination of a calcium channel blocker and a .beta.-blocker such as nifedipine and atenolol; and a combination of a calcium channel blocker and an ACE inhibitor such as felodipine and ramipril;

combinations of an antihypertensive agent and an antidiabetic agent, such as an ACE inhibitor and a sulfonylurea, e.g., irbesartan and glipizide;

combinations of antihistamines and antiasthmatic agents, e.g., an antihistamine and a leukotriene receptor antagonist such as loratadine and zafirlukast, desloratidine and zafirlukast, and cetirazine and montelukast;

combinations of antiinflammatory agents and analgesics, e.g., a COX-2 inhibitor and a nonsteroidal antiinflammatory agent (NSAID) such as rofecoxib and naproxen, or a COX-2 inhibitor and a salicylate such as celecoxib and aspirin;

combinations of an anti-obesity drug and an antidiabetic agent, e.g., a lipase inhibitor such as orlistat in combination with metformin;

combinations of a lipid-regulating agent and a drug for treating coronary artery disease, e.g., fenofibrate and ezetimibe, or lovastatin and ezetimibe; and other combinations, such as docetaxel and cisplatin, tirapazamine and cisplatin, metoclopramide and naproxen sodium, an opioid analgesic such as oxycodone and an anti-inflammatory agent, an agent for treating erectile dysfunction, such as alprostadil, with an antihypertensive/vasodilator such as prazosin.

The aforementioned examples are merely illustrative, and it must be emphasized that any given drug identified by structural or functional class may be replaced with another drug of the same structural or functional class.

Any drug substance(s) may be administered in the form of a salt, ester, hydrate, solvate, coordination complex, coordination compound, amide, pro-drug, active metabolite, isomer, analog, fragment, or the like, provided that the salt, ester, hydrate, solvate, coordination complex, coordination compound, amide, pro-drug, active metabolite, isomer, analog or fragment, is pharmaceutically acceptable and pharmacologically active in the present context. Salts, esters, hydrates, solvates, coordination complexes, coordination compounds, amides, pro-drugs, metabolites, analogs, fragments, and other derivatives of the active agents may be prepared using standard procedures known to those skilled in the art of synthetic organic chemistry and described, for example, by J. March, Advanced Organic Chemistry: Reactions, Mechanisms and Structure, 4th Edition (New York: Wiley-Interscience, 1992).

For example, acid addition salts are prepared from a drug substance in the form of a free base using conventional methodology involving reaction of the free base with an acid. Suitable acids for preparing acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. An acid addition salt may be reconverted to the free base by treatment with a suitable base. Conversely, preparation of basic salts of acid moieties that may be present on an active agent may be carried out in a similar manner using a pharmaceutically acceptable base such as sodium hydroxide, potassium hydroxide, ammonium hydroxide, calcium hydroxide, trimethylamine, or the like. Preparation of esters involves transformation of a carboxylic acid group via a conventional esterification reaction involving nucleophilic attack of an RO.sup.-moiety at the carbonyl carbon. Esterification may also be carried out by reaction of a hydroxyl group with an esterification reagent such as an acid chloride. Esters can be reconverted to the free acids, if desired, by using conventional hydrogenolysis or hydrolysis procedures. Amides may be prepared from esters, using suitable amine reactants, or they may be prepared from an anhydride or an acid chloride by reaction with ammonia or a lower alkyl amine. Prodrugs and active metabolites may also be prepared using techniques known to those skilled in the art or described in the pertinent literature. Pro-drugs are typically prepared by covalent attachment of a moiety that results in a compound that is therapeutically inactive until modified by an individual's metabolic system.

Other derivatives and analogs of the active agents may be prepared using standard techniques known to those skilled in the art of synthetic organic chemistry, or may be deduced by reference to the pertinent literature. In addition, chirally active agents may be in isomerically pure form, or they may be administered as a racemic mixture of isomers.

Another component of the pharmaceutical formulations of the present invention provides at least one water-swellable, pH independent polymer such as the carbohydrate-based polymers including, for example, hypromellose (formerly known as the family of hydroxypropyl methylcellulose), hydroxypropyl ethyl celluloses, hydroxypropyl cellulose, hydroxyethyl cellulose, methyl cellulose or other constituents Grades of these hypromellose copolymers typically used with the present invention include the E and K series such as for example, Dow Chemical Company's (Midland, Mich. USA) or Aqualon's (with a North American presence in Wilmington, Del.) E4M, E10M, K100LV, K4M, K15M, K25M, K100M, K200M and mixtures of various molecular weights and grades. Grades of hydroxyethyl cellulose include, for example, Aqualon's Natrasol® polymers HHX (mol. Wt. 1,300,000), HX (mol. wt. 1,000,000), H (mol. wt. 1,000,000), M (mol. wt. 720,000 and G (mol. wt. 1,150,000), and mixtures thereof. Grades of hydroxypropyl cellulose include, for example, Aqualon's HPC polymers MF and MXF (mol. wt. 580,000) and KF and HXF (mol. wt. 1,150,000), and mixtures thereof. Grades and ethyl cellulose include, for example, Dow Chemical Company's Ethocel® polymers 7FP, 10FP and 100FP and Aqualon's polymers T10EC, N7, N10, N17, N22, N50, N100 and N200, and mixtures thereof. These and all other components, additives, excipients and the like are to be pharmaceutically acceptable.

Another component of the pharmaceutical formulations of the present invention provides at least at least one anionic, pH-dependent, gel-forming copolymer such as a mono-valent alginate salt such as sodium, potassium or ammonium alginate salts, or combinations thereof, and sodium carboxymethyl cellulose and the like, or mixtures of one or more alginate salt and carboxymethyl cellulose and the like. These components are readily available in the commercial market.

Another component of the pharmaceutical formulations of the present invention provides at least at least one polymer selected from the group consisting of a cationic polymer; and a hydrocolloid. The cationic polymer can be, for example, chitosan or a derivative thereof including, for example, trimethylchitosan and quartermised chitosan, and chitosan-derived materials including, for example, those taught in U.S. Pat. No. 5,747,475. Either high or low molecular weight chitosan products can be used in the pharmaceutical formulations of the present invention and are readily available in pharmaceutical grade from suppliers located world-wide. The hydrocolloid used in the formulations of the present invention can be carrageenan. Carrageenans are available as iota, kappa and lambda carrageenans, with iota being used most frequently used and lambda being used least frequently. Various salt forms of carrageenans are also available including, for example sodium carrageenan. Typically used grades of iota carrageenan include, without limitation, carrageenan NF AEP® brand colloids (Hadley, N.Y. USA) FD433 (1% viscosity; 300-400 cps) and FD384 (1% viscosity; about 100 cps). Viscosity of other carrageenan products ranges from about 50 to about 4000 cps.

Ranges of concentration of the components of the present invention will vary depending upon the desired release characteristics of a respective formulation and can be readily adjusted according to known practices.

More specifically, each drug substance is present in the desired amount such that the dosage strength is consistent with labeled or desired concentrations for the appropriate therapeutic index. Considering the range of drug substances that can be used in the formulations of the present invention, the range used will be tailored to that specific drug substance, whether used or in combination with one or more other drug substance.

Generally:

the at least one water-swellable, pH independent polymer is used, whether as an individual polymer or collectively, in the range from about 10 percent to about 90 percent, with other ranges including, for example, from about 20 to about 50 percent, and from about 30 to about 40 percent;

the at least one anionic, pH-dependent, gel-forming copolymer is used, whether as an individual copolymer or collectively, in the range from about 10 percent to about 90 percent with other ranges including, for example, from about 10 to about 50 percent, from about 10 to about 30 percent and from about 15 to about 25 percent; and

the cationic polymer or hydrocolloid, whether used individually or collectively, in the range from about 0.1 percent to about 25 percent with other ranges including, for example, from about 0.5 to about 20 percent and from about 5 to about 15 percent.

However, as noted below, there may be circumstances including, for example, when using poorly soluble drug substances and/or to decrease release times, that the total matrix load in a pharmaceutical formulation of the present invention may be equal to or less than about 30 percent.

An ordinarily skilled artisan will recognize that a number of factors or variables can affect the rate of delivery of drug substance from a matrix of the present invention including, for example, drug substance water solubility, drug substance load/polymer ratio in the formulation, the water solubility and viscosity of the polymers. Using the parameters set forth herein, each drug substance and release profile target should be dealt with on a case by case basis. As a starting point, one formulation of the present invention for a moderate drug substance drug load of about 17%, moderate solubility of the drug substance such as diclofenac potassium with a release over the course of about 12 hours would be represented by the following formulation:

| Ingredient | % w/w |
|---|---|
| Drug substance | 16.7 |
| Hypromellose K100M | 30.0 |
| Na+ Alginate | 25.0 |
| Carrageenan or Chitosan | 10.0 |
| Co-processed | 17.3 |
| microcrystalline | |
| cellulose/colloidal silica | |
| Magnesium Stearate | 1.0 |

To deal with, for example, a highly water soluble drug substance or to extend the release time of a drug substance, modifications to the formulations could include:
Lower drug substance drug load and increase the overall polymer content—typically would require an increased size of the drug product;
Substitution of a less water soluble polymer such as ethylcellulose for the hypromellose.
Increase the molecular weight of the polymers utilized;
Minimize the surface are of the tablet geometry in relation to the volume. Use round shaped tablets;
Reduce the percentage (w/w) of the use of carrageenan and/or chitosan and increase the use of high molecular weight, low solubility polymers; and/or
Avoid the use of water soluble tablet diluents and use insoluble diluents such microcrystalline cellulose. Reduce the level of diluent and increase polymer loading.

To deal with poorly soluble drug substances and/or to decrease release times:
Lower the overall polymer matrix load to as low as 20-30% of the formulation;
Incorporate a water soluble diluent such as lactose;
Minimize or avoid the use of water insoluble polymers such as ethylcellulose and use low molecular weight versions of polymers such as hypromellose and hydroxypropyl cellulose;
Include a surfactant or solublizer in the formulation;
Use micronized drug substance; and/or
Use a multi-particulate minitab system to maximize the surface area to volume ratio of the drug product.

Preparation of the pharmaceutical formulations of the present invention is through conventional means known to the ordinarily skilled artisan in the pharmaceutical formulation arts and include, for example, direct compression, dry granulation and wet granulation. The following general methods of preparing pharmaceutical formulations of the present invention are presented as exemplification and are not intended to limit the formulations of the present invention in any way whatsoever.

Direct compression is accomplished by delumping all of the ingredients, including the drug substance(s) and sieving to a desired range of particle sizes. It may be desirable to delump each ingredient to the same or different size providing the sizes permit blending to homogeneity. The components are then blended, recognizing there may be a need to blend some or almost all of the components in a first blending, followed by a second or subsequent blending(s) of the original ingredients plus additional ingredients. Following appropriate blending, tablets, minitablets (as known to the skilled artisan in the pharmaceutical formulation industry), direct-compressed multi-particulates of one or more sizes and the like may be direct compressed to provide the desired product which may be in the form of a final drug product, filled into capsules or other forms for solid-dose administration, added to one or more additional direct compressed product to form a multi-layered drug product and the like. As such, the term “direct compression” may relate to part or all of a process for preparing pharmaceutical formulations according to the present invention.

One of ordinary skill in the art will recognize that there exist a multitude of methods to accomplish wet granulation as part or all of a process step for the preparation of drug products. Accordingly, each or any of such processes may be used, in part or in whole, for the preparations of pharmaceutical formulations of the present invention. Without limiting the present invention in any way, one commonly used wet granulation process includes, for example, wet top spray granulation. After all ingredients are delumped and sieved to the desired size, the resulting blend of ingredients is added to an appropriate fluid bed processor equipped with a spray gun for fluidizing the blended ingredients using standard practices. The resulting granulation is dried, typically in the fluid bed, milled to a desired range of particle sizes, and used for preparation of a final formulation. One alternative to this process is known as high shear wet granulation. Similarly, the ingredients are sieved or delumped to a desired size and added to an appropriate processor, the blended ingredients are mixed, and frequently chopped while the solvent, typically water or other aqueous-based solvent, is sprayed over the mass during granulation. The wet granulation is typically fluidized in a fluid bed then dried, milled (frequently with the addition of additional desired ingredients). Alternatively, low shear wet granulation can also be used depending upon the equipment available, ingredients being used and the desired outcome. The product of a wet granulation process can be formed into tablets, minitablets, direct-compressed multi-particulates of one or more sizes and the like and which may be in the form of a final drug product, filled into capsules or other forms for solid-dose administration, added to one or more additional direct compressed product to form a multi-layered drug product and the like, as desired.

The ordinarily skilled artisan will also recognize that there exist a multitude of methods to accomplish dry granulation as part or all of a process step for the preparation of drug products. Dry granulation frequently is used to improve the flow or other characteristic of a final blend of ingredients to be formed into a final drug product. Accordingly, each or any of such processes may be used, in part or in whole, for the preparations of pharmaceutical formulations of the present invention. Without limiting the present invention in any way, one commonly used dry granulation process includes, for example, delumping and/or sieving the desired ingredients, blending ingredients and feeding the ingredients through, for example, a roller compactor that produces a ribbon of compressed product, then milling the resulting ribbon. The milled product may then be compressed as set forth above or further blended with additional ingredients and then compressed.

Pharmaceutical formulations according to the present invention that are in tablet form should be compressed to a sufficient hardness to prevent the premature ingress of the aqueous medium and prevention of surface pitting and breakage during coating of the core, when applicable. When manufacturing tablets, the complete mixture, in an amount sufficient to make a uniform batch of tablets, is subjected to tableting in a conventional tableting machine at an appropriate pressure. Typical compression forces are about 5 to about 50 kilo Newtons (kN).

Other optional ingredients, those that are typically used in pharmaceuticals, may also be used in the present pharmaceutical formulations. These include, for example, fillers, lubricants, glidants, coloring agents, anti-oxidizing agents, and the like, the use of each as known to the ordinarily skilled artisan. The following are provided for the purpose of example, only, and are not intended to limit in any way the scope of the present invention. Fillers include, for example, sugars, which include dextrose, sucrose, maltose, and lactose, sugar-alcohols, which include mannitol, sorbitol, maltitol, xylitol, starch hydrolysates, which include dextrins, and maltodextrins, and the like, microcrystalline cellulose or other cellulosic derivatives, dicalcium phosphate, tricalcium phosphate and the like, and mixtures thereof. Typical amount of fillers used in a drug product may be as low as zero when not required or desired, and may be as high as 50 percent (w/w) for highly active, low dosage drug substances.

Lubricants include, for example, long chain fatty acids and their salts, such as magnesium stearate and stearic acid, talc, glycerides and waxes. Typical amounts of lubricants used in a drug product can range from about 0.1 to about three percent (w/w).

Glidants include, for example, colloidal silicon dioxide, talc and the like. Typical amounts of glidants used in a drug product can range from about 0.1 to about one percent (w/w).

Coloring agents include, for example, FD&C colors such as FD&C Yellow No. 6, FD&C Red No. 2, FD&C Blue No. 2, food lakes and the like. Typical amounts of coloring agents used in a drug product can range from about 0.1 to about one percent (w/w).

Anti-oxidants include, for example, ascorbic acid, sodium metabisulphite and the like. Typical amounts of anti-oxidants used in a drug product can range from about 0.1 to about one percent (w/w).

The pharmaceutical formulations of the present invention can be coated with one or more coatings for a variety of purposes. Generally, various coatings used with pharmaceutical dosage forms include, for example, enteric coatings, seal coatings, film coatings, barrier coatings, compress coatings, fast disintegrating coatings, and enzyme degradable coatings. Multiple coatings can be applied for desired performance. Further, the dosage form can be designed for immediate release, pulsatile release, multi-modal release, delayed release, targeted release, synchronized release, or targeted delayed release. These terms, and techniques to achieve each, are well known in the pharmaceutical art. For release and/or absorption control, the present pharmaceutical formulations can be made with various types and levels or thicknesses of coats and can be partially or completely covered by a respective coating. Such coatings may be added with or without a drug substance. When one or more drug substance is added to a coating, such drug substance may be the same or different than the at least one drug substance included in the matrix of a pharmaceutical formulation of the present invention.

When formulated as a capsule using the pharmaceutical formulations of the present invention, the capsule can be a hard or soft capsule made from any pharmaceutically-acceptable and appropriate material.

Coatings, as referenced above and otherwise, are known in the art, but for clarity, the following brief descriptions are provided:

Seal coating, or coating with isolation layers (pharmaceutically non-functional coatings): Thin layers of up to 20 microns in thickness can be applied for variety of reasons including, for example, particle porosity reduction, to reduce dust, for chemical protection, to mask taste, to reduce odor, to minimize gastrointestinal irritation and the like. The isolating effect is proportional to the thickness of the coating. Water soluble cellulose ethers are commonly used for this application. HPMC and ethyl cellulose in combination, or Eudragit® E100 (Evonik Rohm GmbH, Darmstadt, Germany), are commonly used for taste masking applications.

Pharmaceutically functional coatings include, for example, enteric coatings: The term “enteric coating” as used herein relates to a mixture of pharmaceutically acceptable excipients which is applied to, combined with, mixed with or otherwise added to the carrier or composition, typically to achieve delayed release of one or more drug substances in a drug product. The coating(s) may be applied to a tablet, a capsule, and/or pellets, beads, minitablets, granules or particles of the present pharmaceutical formulation. The coating may be applied through an aqueous dispersion or after dissolving in appropriate solvent. Additional additives and their levels, and selection of a primary coating material or materials will depend on the following properties:

1. resistance to dissolution and disintegration in the stomach;

2. impermeability to gastric fluids while in the stomach;

3. ability to dissolve or disintegrate in a desired fashion at the target intestine site;

4. physical and chemical stability of a drug product during storage;

5. non-toxicity;

6. easy application as a coating (substrate friendly); and

7. economical practicality.

To achieve a delayed-release affect, any coating(s) should be applied to a sufficient thickness such that the entire coating does not dissolve in the gastrointestinal fluids at pH below about 5, but does dissolve at pH about 5 and above. It is expected that any anionic polymer exhibiting a pH-dependent solubility profile can be used as an enteric coating in the practice of the present invention to achieve delivery of one or more drug substance to the lower gastrointestinal tract. Non-limiting examples of coating used to prepare a delayed-release drug product include:

Shellac, a refined product obtained from the resinous secretion of an insect. This coating dissolves in media of about pH 7 and greater.

Acrylic polymers. The performance of acrylic polymers (primarily their solubility in biological fluids) can vary based on the degree and type of substitution. Examples of suitable acrylic polymers include methacrylic acid copolymers and ammonio methacrylate copolymers. The Eudragit series E, L, S, RL, RS and NE are available as solubilized in organic solvent, aqueous dispersion, or dry powders. The Eudragit series RL, NE, and RS are insoluble in the gastrointestinal tract but are permeable and are used primarily for extended release. The Eudragit series E dissolve in the stomach. The Eudragit series L, L-30D and S are insoluble in stomach and dissolve in the intestine.

Cellulose Derivatives. Suitable cellulose derivatives include, for example, ethyl cellulose; reaction mixtures of partial acetate esters of cellulose with phthalic anhydride (the performance can vary based on the degree and type of substitution; cellulose acetate phthalate (CAP) dissolves in pH >6; Aqucoat® CMP (FMC, Philadelphia, Pa. USA) is an aqueous based system; cellulose acetate trimellitate; methylcellulose; hydroxypropylmethyl cellulose phthalate (HPMCP; the performance can vary based on the degree and type of substitution; grades include, for example HP-50, HP-55, HP-55S, HP-55F); hydroxypropylmethyl cellulose succinate (HPMCS) (the performance can vary based on the degree and type of substitution; grades include, for example, AS-LG (LF), which dissolves at about pH 5, AS-MG (MF), which dissolves at anout pH 5.5, and AS-HG (HF), which dissolves at higher pH. These polymers are offered as granules, or as fine powders for aqueous dispersions;

Poly Vinyl Acetate Phthalate (PVAP). PVAP dissolves in about pH 5 and greater, and it is much less permeable to water vapor and gastric fluids; and

Combinations of the above materials can also be used.

The coating can, and usually does, contain a plasticizer and possibly other coating excipients such as colorants, talc, and/or magnesium stearate, which are well known in the art. Suitable plasticizers include, for example: triethyl citrate, glyceryl triacetate, acetyl triethyl citrate, polyethylene glycol 400, diethyl phthalate, tributyl citrate, acetylated monoglycerides, glycerol, fatty acid esters, propylene glycol, and dibutyl phthalate. More particularly, anionic carboxylic acrylic polymers usually contain 10-25% by weight of a plasticizer, especially dibutyl phthalate, polyethylene glycol, triethyl citrate and triacetin.

Conventional coating techniques such as spray or pan coating are employed to apply coatings. The coating thickness should be sufficient to ensure that the oral dosage form remains intact until the desired site of topical delivery in the lower intestinal tract is reached.

Colorants, detackifiers, surfactants, antifoaming agents, lubricants, stabilizers such as hydroxypropylcellulose, acid/base may be added to the coatings besides plasticizers to solubilize or disperse the coating material, and to improve coating performance and the coated drug product.

It is to be understood and expected that variations in the principles of invention herein disclosed may be made by one skilled in the art and it is intended that such modifications are to be included within the scope of the present invention.

### Experimental Details

The following dissolution parameters were used for all Examples, except for Examples 3, 19-20 and 21-22:

USP Apparatus II

Paddle speed 50 rpm

Temperature 37° C.

HPLC analysis of samples

The paddle speed for Examples 19-20 was 50 rpm for all time points except for infinity wherein the paddle speed was 250 RPM for an additional fifteen minutes. Examples 21-22 used cylindrical baskets spinning at 100 rpm.

The following dissolution media were used for the respective Example:

| Tablet | Example | Disso Media |
|---|---|---|
| Minocycline HCl | 1, 12 | 0.1 N HCl, pH 4.5 acetate300 mM, |
| | | pH 6.8 phosphate (50 mM) |
| 1-Methylnicotinamide | 2, 13 | pH 6.8 phosphate (50 mM) |
| Diclofenac potassium | 4, 6 | pH 6.8 phosphate (50 mM) |
| Nifedipine | 5 | 0.1N HCl + 1% sodium lauryl sulfate |
| Acetaminophen | 7, 8, 9 | pH 6.8 phosphate, pH 4.5 acetate |
| Nifedipine | 10, 11 | 0.1N HCl + 1% SLS, pH 6.8 |
| (unmicronized) | | phosphate + 1% SLS, pH 4.5 |
| | | Acetate + 1% SLS, 0.1N HCL + 2% |
| | | SLS, 0.1N HCl + 2% CTAB |
| Morphine Sulfate | 19-20 | 0.1N HCl |
| Oxycodone HCl | 21-22 | Simulated gastric fluid |
| | | (without enzymes) |

### Example 1

### Direct Compression of 50 mg Minocycline HCl Tablets

A one kilogram batch to produce 50 mg strength minocycline hydrochloride tablets was prepared using direct compression. The following formulation was utilized:

| Ingredient | % w/w |
|---|---|
| Minocycline HCl | 16.7 |
| Hypromellose K100M | 30.0 |
| Na+ Alginate | 25.0 |
| Carrageenan FD433 | 10.0 |
| Co-processed | 17.3 |
| microcrystalline | |
| cellulose/colloidal silica | |
| (Prosolve HD90 ®; JRS | |
| Pharma, Patterson, NT | |
| USA) | |
| Magnesium Stearate | 1.0 |

Tablet weight 327 mg

All the ingredients were delumped prior to use with a 20 mesh screen except for the magnesium stearate which was passed through a 40 mesh sieve. The ingredients minus the magnesium stearate were charge to a 4 quart v-blender and blended for a period of five minutes. The magnesium stearate was charge to the blender and blending was continued another three minutes. The blend was compressed on a three station Korsch PH105 tablet press equipped with ⅜″ diameter round standard concave tablet tooling producing tablets with a weight of ˜327 mg, ˜8 kp hardness and a thickness of ˜0.187″.

### Example 2

### Dry Granulation of 270 mg 1-MNA Tablets and Coating to Delay Release

To improve the flow of the final blend used to compress tablets, a dry granulation method was utilized in this example.

| Ingredient | % w/w |
|---|---|
| 1-methylnicotinamide | 33.7 |
| chloride | |
| Hypromellose K100M | 40.0 |
| Na+ Alginate | 20.0 |
| Chitosan M | 0.5 |
| Co-processed | 4.8 |
| microcrystalline | |
| cellulose/colloidal silica | |
| Magnesium Stearate | 1.0 |

Tablet weight 800 mg

All the ingredients for a 2 kg batch were delumped prior to use with a 20 mesh screen except for the magnesium stearate which was passed through a 40 mesh sieve. The ingredients plus twenty-five percent of the total magnesium stearate quantity was charged to a 8 quart v-blender and blended for a period of ten minutes. The blend was passed through a Vector TF Mini roll compactor equipped with serrated rolls using a roll speed of 3 rpm, feed screw speed 5 rpm, and a compaction force of 2.0 tons. A 0.040″ thick ribbon was produced which was milled to size using a Quadro Comil equipped with a 0.040″ grater type screen. The milled ribbons were charged to v-blender along with the remaining three quarters of the magnesium stearate and blended for three minutes. The blend was compressed on a three station Korsch PH105 tablet press equipped with ⅜″ diameter round standard concave tablet tooling producing tablets with a weight of ˜800 mg, ˜10 kp hardness and a thickness of ˜0.268″.

Delayed Release Coating for the Product of Example 2

The release of the 1-methylnicotinamide from the matrix tablet was targeted to be a delayed release of approximately 2 hours followed release over the course of 12-24 hours. The 40% hypromellose base matrix tablet provided the extended release of approximately 12 hours as desired, but without a delay in the release of the drug substance without a coating. A coating on the tablet was employed to delay the release of the 1-methylnicotinamide from the tablet. The strategy was to utilize the nature of the tablet to swell as it became hydrated. Applying a semi-water permeable coating to the tablet delays the intrusion of water into the tablet and thus the swelling. Eventually, enough water penetrates the coating causing swelling and pressure buildup with a subsequent rupture of the coating. Upon rupturing, the tablet begins releasing the drug substance as a matrix tablet. The delay is controlled by the thickness of the coating applied to the tablet and/or the water permeability of the coating applied. Ethyl cellulose (Colorcon Surelease®; West Point, Pa. USA) was chosen as the semi-permeable coating with the permeability increased by incorporating a low molecular weight, low viscosity pore forming agent hypromellose (Dow E5LV).

The 270 mg methylnicotinamide matrix tablet manufactured by direct compression was coated (with aid of placebo shams to bulk up the coating pan load to 8 kg) in an Accelacota 24″ coating pan equipped with 2 spray guns. A 1% weight gain of a seal coating of the non-functional coating Opadry® II White (Colorcon formula #57U18539) was applied to the tablets to help prevent tablet erosion and coating peeling problems. The Opadry® was applied to the tablet cores in the coating pan using the following parameters:

| | |
|---|---|
| Inlet air temp ~75° C. | Inlet Airflow ~200 cfm |
| Spray rate: 40 g/min | Pan Speed: 12 rpm |
| Bed Temp ~45° C. | Gun-bed distance: 5″ |
| Outlet air temp ~45° C. | Coating Suspension: 15% solids |
| Amount sprayed: 533 g | Pan Load 8 kg (0.5 kg active, 7.5 kg placebo) |

The semi-permeable coating was manufactured by mixing 20 g of hypromellose E5LV in 900 g of Milli-Q water in a stockpot equipped with a propeller stirrer. A 1227 g aliquot of ethyl cellulose based Surelease® 19040 suspension (contains 25% solids) was charged to the stirring hypromellose solution, bringing the solids content to 15%.

The ethyl cellulose based Surelease® modified with 5% of the Dow E5LV hypromellose was applied to the seal coated tablets using the following processing parameters:

| | |
|---|---|
| Inlet air temp ~75° C. | Inlet Airflow ~200 cfm |
| Spray rate: 40 g/min | Pan Speed: 12 rpm |
| Bed Temp ~50° C. | Gun-bed distance: 5″ |
| Outlet air temp ~50° C. | Coating Suspension: 15% solids |
| Amount sprayed: 2090 g | Pan Load: 8 kg (0.5 kg active, 7.5 kg placebo) |

Samples of tablets were pulled with a 3% and 4% weight gain of the modified Surelease® coating. The coated tablets were dried/cured for 18 hours at 40° C. in ambient atmosphere in an oven. The release of the 1-methylnicotinamide was delayed 1-2 hours depending upon the coating amount. In addition, the release of the coated product produced a more linear release profile versus the uncoated tablet.

### Example 3

### Aqueous Wet Top Spray Granulation in Fluid Bed

In this example, fluidized bed top spray granulation is utilized to manufacture 50 mg strength nifedipine tablets. All the ingredients of a 2 kg batch except for the magnesium stearate are screened through a 20 mesh sieve and charged to a Niro MP-1 fluid bed processor equipped with a spray gun for top spraying.

| Ingredient | % w/w |
|---|---|
| Nifedipine | 16.7 |
| K100M | 30.0 |
| Na+ Alginate | 25.0 |
| Chitosan M | 10.0 |
| Co-processed | 17.3 |
| microcrystalline | |
| cellulose/colloidal silica | |
| Magnesium Stearate | 1.0 |

300 mg tablets

The materials are fluidized with an inlet air temperature of 65° C. and water is sprayed at 30/g/minute at 30 psi atomization pressure. A total of 450 g of water is sprayed. The granulation is dried to an LOD of ˜2.0% in the fluid bed. The dried granulation is milled to size using a Quadro Comil equipped with a 0.040″ grater type screen. The milled granulation is charged to v-blender along with the magnesium stearate and blended for three minutes. The blend is compressed on a three station Korsch PH105 tablet press equipped with ⅜″ diameter round standard concave tablet tooling producing tablets with a weight of ˜300 mg, ˜8 kp hardness.

### Example 4

### Wet Granulation—Aqueous High Shear 50 mg Diclofenac Potassium Tablets

| Ingredient | % w/w |
|---|---|
| Diclofenac Potassium | 16.7 |
| K100M | 30.0 |
| Na+ Alginate | 25.0 |
| Carrageenan | 10.0 |
| Co-processed | 17.3 |
| microcrystalline | |
| cellulose/colloidal silica | |
| Magnesium Stearate | 1.0 |

300 mg tablets

High shear aqueous granulation was utilized. All the ingredients used for a 1 kg batch except for the magnesium stearate (screened through a 40 mesh sieve) were screened through a 20 mesh sieve and charged to a Niro PP-1 high shear granulator. The materials were mixed for three minutes with an impeller speed of 300 rpm and no chopper. With the impeller running at 300 rpm and the chopper set at low speed of 1500 rpm, 350 g of water was sprayed onto the stirring mass over the course of approximately 3 minutes. An additional 1 minute of mixing was utilized to produce a granulation. The wet granulation was fluidized in a Niro MP-1 fluidized bed with an inlet air temperature of 65° C. and dried to an LOD of ˜2.2% in the fluid bed. The dried granulation was milled to size using a Quadro Comil equipped with a 0.050″ grater type screen. The milled granulation was combined with the magnesium stearate and bag blended for three minutes. The blend was compressed on a three station Korsch PH103 tablet press equipped with ⅜″ diameter round standard concave tablet tooling producing tablets with a weight of ˜300 mg, ˜10 kp hardness.

### Example 5

### 50 mg Nifedipine Tablets and Multiparticulate Capsules Containing Mini-Tablets Manufactured Using a Low Shear Wet Granulation with and without a Surfactant

Nifedipine tablets and minitablets were manufacture using micronized nifedipine with and without the SLS surfactant per the formulations shown below.

| | % w/w | | |
|---|---|---|---|
| | | 01108-039 | 01108-040 |
| | Lot 01108-037 | (15 mini-tablets | (15 mini-tablets |
| | (300 mg tablets | w/o surfactant in | with surfactant |
| Ingredient | with surfactant) | a capsule) | in a capsule) |
| Micronized | 16.2 | 16.7 | 16.2 |
| Nifedipine | | | |
| Hypromellose | 29.1 | 30.0 | 29.1 |
| K100M | | | |
| Sodium alginate | 24.3 | 25.0 | 24.3 |
| Carrageenan | 9.7 | 10.0 | 9.7 |
| Prosolve HD90 | 16.8 | 17.3 | 16.8 |
| Sodium lauryl | 2.9 | — | 2.9 |
| sulfate | | | |
| Magnesium | 1.0 | 1.0 | 1.0 |
| Stearate | | | |
| Total | 100.0 | 100.0 | 100.0 |
| | 300 mg tablet | 300 mg of minitabs | 300 mg of |
| | | | minitabs |

In these examples the ingredients (except for the magnesium stearate) for a 100 g batch were screened through a 20 mesh sieve and charged to a Kitchen Aide planetary type mixer and mixed for 1 minute. Either 50 g of water or 53 g of 6% sodium lauryl sulfate. (SLS) in water was slowly poured into the mixing materials over the course of about 5 minutes. The granulation was then spread out in a stainless steel tray and dried in an oven for approximately 245 hours at 50° C. to an LOD moisture content of 2-3%. The dried granulations (with or without SLS) were milled with a Comil using a square style impeller and a 0.050″ grater type screen. Magnesium stearate was screened through a 40 mesh sieved and 1% delumped magnesium stearate was bag blended into each granulation with 72 tumbles. Tablets (⅜″ round standard concave) were compressed from the granulation containing SLS at a target tablet weight of 300 mg, and hardness of 8 kp.

Mini-tablets, from both granulations with and without SLS, were compressed using 0.0984″ diameter round standard concave tooling at a target weight of ˜20 mg and a hardness of 3 kp. Fifteen minitabs per capsule (300 mg fill weight) were placed in size 1 hard gelatin capsules to provide a multi-particulate system.

### Example 6

### Diclofenac Tablets prepared by Direct Compression and High Sheer Wet Granulation

Diclofenac Potassium 50 mg Strength Tablet: lots 003 A, B, D, E were prepared by direct compression as used in Example 1 and lot 041 was prepared by High Shear Wet Granulation as used in Example 4.

| | % w/w | | | | |
|---|---|---|---|---|---|
| Ingredient | A | B | D | E | 041 |
| Diclofenac K+ | 16.7 | 16.7 | 16.7 | 16.7 | 16.7 |
| K100M | 30.0 | 30.0 | 30.0 | 30.0 | 30.0 |
| Na+ Alginate | 25.0 | 25.0 | 25.0 | 25.0 | 25.0 |
| Chitosan L | 10.0 | — | — | — | |
| Chitosan M | — | 10.0 | — | — | |
| Carrageenan | — | — | — | 10.0 | |
| FD384 | | | | | |
| Carrageenan | — | — | 10.0 | — | |
| FD433 | | | | | |
| Carrageenan | — | — | — | — | 10.0 |
| Viscarin | | | | | |
| Prosolve HD90 | 17.3 | 17.3 | 17.3 | 17.3 | 17.3 |
| Magnesium | 1.0 | 1.0 | 1.0 | 1.0 | 1.0 |
| Stearate | | | | | |

300 mg tablet weight

### Example 7

### Acetaminophen (50 mg Strength Tablets) lots 011 A-E Prepared by Direct Compression as Used in Example 1

| | % w/w | | | | |
|---|---|---|---|---|---|
| Ingredient | A | B | C | D | E |
| APAP | 16.7 | 16.7 | 16.7 | 16.7 | 16.7 |
| K100M | 30.0 | 30.0 | 30.0 | 30.0 | 30.0 |
| Na+ Alginate | 25.0 | 25.0 | 25.0 | 25.0 | 25.0 |
| Chitosan L | 10.0 | — | — | — | — |
| Chitosan M | | 10.0 | — | — | — |
| Carrageeenan | — | — | — | 10.0 | — |
| FD384 | | | | | |
| Carrageenan | — | — | 10.0 | — | — |
| FD433 | | | | | |
| Prosolve HD90 | 17.3 | 17.3 | 17.3 | 17.3 | 17.3 |
| Magnesium | 1.0 | 1.0 | 1.0 | 1.0 | 1.0 |
| Stearate | | | | | |

300 mg tablet

### Example 8

### Acetaminophen (50 mg Strength Tablets) lots 013 A-B Prepared by Direct Compression as Used in Example 1

% w/w

| Ingredient | A | B |
|---|---|---|
| APAP | 14.3 | 16.7 |
| K100M | 30.0 | 30.0 |
| K100LV | — | 5.0 |
| Na+ Alginate | 25.0 | 25.0 |
| Dibasic Calcium | 10.7 | 5.7 |
| Phosphate Dihydrate | | |
| Carrageenan FD433 | 10.0 | 10.0 |
| Prosolve HD90 | 10.0 | 10.0 |
| Magnesium Stearate | 1.0 | 1.0 |

350 mg tablet

### Example 9

### Delayed Release Coated Acetaminophen (50 mg Strength Tablets)

Lots 011 and 13 tablets were prepared by direct compression as used in Example 1 and coated with a hypromellose seal coat as used in Example 2 of a 1% weight gain followed by a 2, 3, or 4% weigh coat of ethyl cellulose/hypromellose (a semi-permeable coating). The tablets were assigned lots 033 −½%, −⅓% or −¼% depending on coating amounts.

### Example 10

### Nifedipine (50 mg Strength Tablets) Tablet were Prepared by Direct Compression as Used in Example 1

% w/w

| Ingredient | 017 | 020 |
|---|---|---|
| Nifedipine | 16.7 | 16.7 |
| (un-micronized) | | |
| K100M | 30.0 | 30.0 |
| Na+ Alginate | 25.0 | 25.0 |
| Chitosan M | 10.0 | — |
| Carrageenan FD433 | — | 10.0 |
| Prosolve HD90 | 17.3 | 17.3 |
| Magnesium Stearate | 1.0 | 1.0 |

300 mg tablet

### Example 11

### Nifedipine (Micronized) Tablets lot 037 and Mini-Tablets lot 039 (with and Internal Surfactant) and Mini-Tablets lot 040 (without and Internal Surfactant). All Tablets were Prepared with a Low Shear Wet Granulation as used in Example 5

| | % w/w | | |
|---|---|---|---|
| | | Lot 039 | Lot 040 |
| | Lot 037 | (15 mini- | (15 mini- |
| | (300 mg | tablets w/o | tablets with |
| | tablets with | surfactant in a | surfactant in a |
| Ingredient | surfactant) | capsule) | capsule) |
| Micronized | 16.2 | 16.7 | 16.2 |
| Nifedipine | | | |
| Hypromellose | 29.1 | 30.0 | 29.1 |
| K100M | | | |
| Sodium alginate | 24.3 | 25.0 | 24.3 |
| Carrageenan | 9.7 | 10.0 | 9.7 |
| Prosolve HD90 | 16.8 | 17.3 | 16.8 |
| Sodium lauryl | 2.9 | — | 2.9 |
| sulfate | | | |
| Magnesium | 1.0 | 1.0 | 1.0 |
| Stearate | | | |
| Total | 100.0 | 100.0 | 100.0 |
| | 300 mg tablet | 300 mg of minitabs | 300 mg |
| | | | of minitabs |

### Example 12

### Minocycline HCl 50 mg Strength Tablets (Lot 022) were Prepared by the Direct Compression Used in Example 1

| Ingredient | % w/w |
|---|---|
| Minocycline HCl | 16.7 |
| K100M | 30.0 |
| Na+ Alginate | 25.0 |
| Carrageenan FD433 | 10.0 |
| Prosolve HD90 | 17.3 |
| Magnesium Stearate | 1.0 |

Tablet weight 327 mg

### Example 13

### 1-Methylnicotinamide Chloride 270 mg Strength Tablets with lots 005 and 018 were Prepared by Direct Compression as Used in Example 1 and lot 009A was Prepared by Dry Granulation as Use in Example 2

| | lot 005 | lot 018 | lot 009A |
|---|---|---|---|
| Ingredient | % w/w | % w/w | % w/w |
| 1-Methylnicotinamide | 33.7 | 33.7 | 27.0 |
| K100M hypromellose | 40.0 | — | 5.0 |
| HPC HXF | — | 40.0 | — |
| Ethocel Standard FP 100 | — | — | 51.5 |
| ethyl cellulose | | | |
| Na+ Alginate | 20.0 | 20.0 | 10.0 |
| Chitosan M | 0.5 | 0.5 | 0.5 |
| Prosolve HD90 | 4.8 | 4.8 | — |
| Magnesium Stearate | 1.0 | 1.0 | 1.0 |
| Tablet weight | 800 mg | 800 mg | 1000 mg |

### Example 14

### Preparation of the Pre-Granulated “Slow” Release Blend

The pre-granulated slow-release blend was prepared as a 1 kg batch using high shear granulation. The polymers were charged to a Niro PP-1 granulator and mixed with 700 g of water at an impeller speed of 300 rpm and a slow chopper setting over the course of sixteen minutes. Mixing was extended an addition three minutes after the water was charged. The granulation was dried in a Niro MP-1 fluid bed processor at an inlet air temperature of 55-65° C. for 30 minutes to a LOD value of 4.8% (the baseline moisture value was 7.9% prior to granulation). The granulation was passed through a Quadro® Comil® equipped with either a 075R or 055R round hole screen at 50% speed.

| Ingredient | % w/w | Amount (g) per kg | |
|---|---|---|---|
| K100M hypromellose | 50 | 0.500 | |
| Sodium Alginate | 41.7 | 0.417 | |
| Carrageenan | 8.3 | 0.083 | |
| Purified Water | N/A | | |
| Total | 100% | 1.0 | kg |

### Example 15

### Preparation of a Pre-Granulated “Fast” Release Blend

The polymers were charged to a Niro PMA 65 high shear granulator and pre mixed with a slow impeller speed and no chopper for three minutes. A total of 10500 g Of water was then sprayed at 650 g/minute onto the polymers while mixing at slow impeller and slow chopper speed. The wet polymers were mixed an additional 3 minutes and then transfered to a Niro MP-3 fluid bed dryer and dried at with an inlet air temperature of 70° C. and air volume of approximately 200CMH. The polymers were dried to a moisture content of 3.4% as determined by LOD. The dried granules were milled in a model 197S Quadro Comil with a round shaped impeller and a 055R (0.055″ in diameter round hole screen) screen at an impeller speed of 30%.

| | | Theoretical |
|---|---|---|
| | | Amount to be |
| % w/w | Component | Dispensed (g) |
| 36.4 | Hypromellose Type 2910 | 5,640 |
| | (Methocel ™ E50 Premium LV) | |
| 45.5 | Sodium Alginate | 6,825 |
| | (Protanal ® LF 120 M) | |
| 18.2 | Carrageenan | 2,730 |
| | (Viscarin GP-209) | |
| NA | Purified Water¹ | 10,500¹ |
| | Port # | |
| 100.0% | Total Amount | 10,000 |

¹Purified water is removed during processing and not considered part of the formula. Purified Water expiry period is 24 hours after dispensing purified water.

### Examples 16-18

### Preparation of 15, 30 and 60 mg Fast-Release Morphine Sulfate Extended Release Tablets

These formulations were prepared via bag blending and direct compression processes. The appropriate amount of active pharmaceutical ingredient, the components shown for the fast-release blend in Example 15 and ProSolv HD 90 were added to an appropriate bag and blended for 120 tumbles. Such components from Example 15 were direct compressed rather than pregranulated. The magnesium stearate was disaggregated using a 40 mesh sieve and blended with the aforementioned blend for an additional 72 tumbles. Tooling, hardness and thickness for the various tablets were as follows:

| | | | Thickness |
|---|---|---|---|
| | Tooling | Hardness (kp) | (inches) |
| 15 mg | 0.2500″ round standard concave | 6 | 0.126 |
| 30 mg | 0.3125″ round standard concave | 10 | 0.161 |
| 60 mg | 0.2500 × 0.4650 arc-diamond | 16 | 0.279 |

| Materials | mg/tablet | | mg/tablet | | mg/tablet | | g/batch | |
|---|---|---|---|---|---|---|---|---|
| Morphine Sulfate, | 15.00 | | 30.00 | | 60.0 | | 1666.7 | |
| USP | | | | | | | | |
| Fast Release | 24.77 | | 49.55 | | 99.1 | | 2752.8 | |
| Blend | | | | | | | | |
| Microcrystalline | 50.00 | | 100.00 | | 200.0 | | 5555.6 | |
| Cellulose and | | | | | | | | |
| Colloidal Silicon | | | | | | | | |
| Dioxide | | | | | | | | |
| (ProSolv ® | | | | | | | | |
| HD 90) | | | | | | | | |
| Magnesium | 0.23 | | 0.45 | | 0.9 | | 0.25 | |
| Stearate (Kosher | | | | | | | | |
| Passover, | | | | | | | | |
| HyQual ®) | | | | | | | | |
| Vegetable Source | | | | | | | | |
| Product Code | | | | | | | | |
| 2257 | | | | | | | | |
| Total | 90.00 | mg | 180.00 | mg | 360.0 | mg | 10,000.0 | g |

### Examples 19-20

### Preparation of 100 and 200 mg Fast-Release Morphine Sulfate Extended Release Tablets

These formulations were prepared via bag blending and direct compression processes. The appropriate amount of active pharmaceutical ingredient, the slow-release blend from Example 14 or the components shown for the fast-release blend in Example 15, respectively, and ProSolv HD 90 were added to an appropriate bag and blended for 120 tumbles. Such components from Example 15 were direct compressed rather than pre-granulated. The magnesium stearate was disaggregated using a 40 mesh sieve and blended with the aforementioned blend for an additional 72 tumbles. Tooling, hardness and thickness for the various tablets were as follows:

| | | | Thickness |
|---|---|---|---|
| | Tooling | Hardness (kp) | (inches) |
| 100 mg | 0.2438 round standard concave | 10 | 0.202 |
| 200 mg | 0.4375″ round standard concave | 10 | 0.260 |

| Materials | mg/tablet | | mg/tablet | | g/batch | |
|---|---|---|---|---|---|---|
| Morphine Sulfate, USP | 100.00 | | 200.0 | | 3333.3 | |
| Fast Release Blend | 82.50 | | 165.0 | | 2750.0 | |
| Microcrystalline Cellulose and | 116.75 | | 233.5 | | 3891.7 | |
| Colloidal Silicon Dioxide | | | | | | |
| (ProSolv ® HD 90) | | | | | | |
| Magnesium Stearate (Kosher | 0.75 | | 1.5 | | 0.25 | |
| Passover, HyQual ®) Vegetable | | | | | | |
| Source Product Code 2257 | | | | | | |
| Total | 300.00 | mg | 600.0 | mg | 100.0 | g |

### Examples 21-22

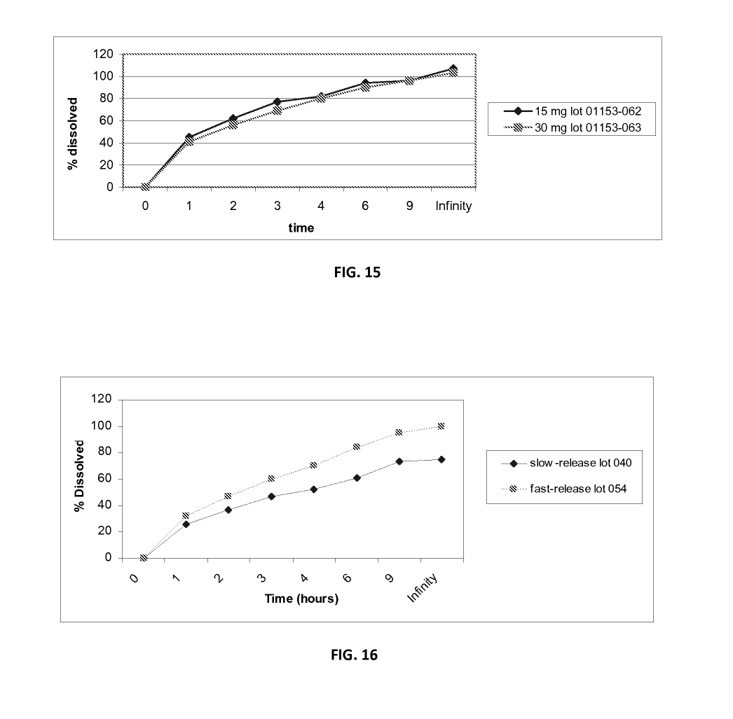

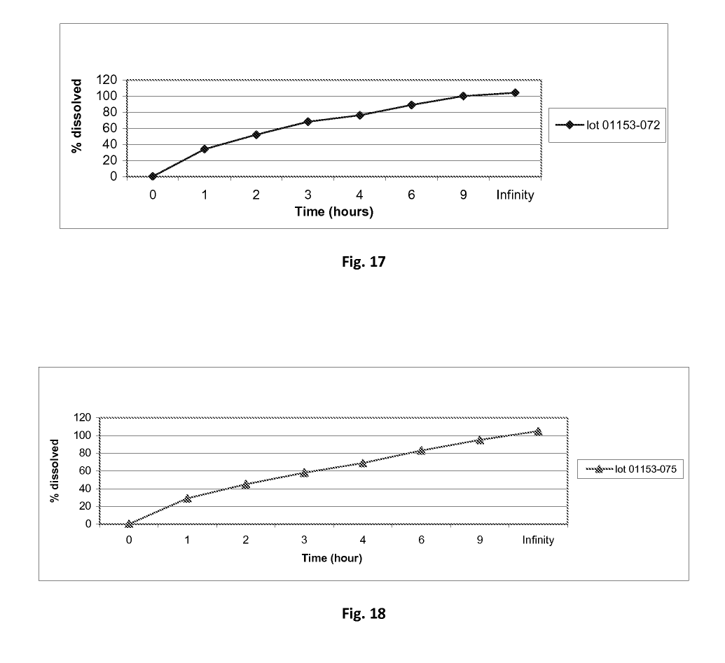

### Preparation of 60 mg Slow-Release and Fast-Release Morphine Sulfate Extended Release Tablets Used for Dissolution Testing as Shown in FIG. 15

These formulations were prepared via bag blending and direct compression processes. The appropriate amount of active pharmaceutical ingredient, the respective slow- or fast-release blends from Examples 15 and 16, respectively, and ProSolv HD 90 were added to an appropriate bag and blended for 120 tumbles. The magnesium stearate was disaggregated using a 40 mesh sieve and blended with the aforementioned blend for an additional 72 tumbles. Tooling, hardness and thickness for the various tablets were as follows:

| | | Hardness | Thickness |
|---|---|---|---|
| | Tooling | (kp) | (inches) |
| 60 mg (slow) | 0.375″ round standard concave | 17 | 0.193 |
| 60 mg (fast) | 0.2500 × 0.4650 arc diamond | 16 | 0.279 |

| | Slow-release | |
|---|---|---|
| | Formulation | Fast-release |
| | Lot 01153-040 (% | Formulation |
| Material | w/w) | Lot 01153-054 (% w/w) |
| Morphine Sulfate, USP | 16.7 | 16.7 |
| Slow Release Blend | 60.0 | NA |
| Fast Release Blend | NA | 27.5 |
| Microcrystalline | 18.0 | 55.6 |
| Cellulose and Colloidal | | |
| Silicon Dioxide | | |
| (ProSolv HD90) | | |
| Magnesium Stearate | 0.25 | 0.25 |

### Examples 23-24

### Preparation of 40 mg Slow-Release and Fast-Release Oxycodone HCl Extended Release Tablets

These formulations was prepared via bag blending and direct compression processes. The appropriate amount of active pharmaceutical ingredient, the respective slow- or fast-release blends from Examples 15 and 16, respectively, and ProSolv HD 90 were added to an appropriate bag and blended for 120 tumbles. The magnesium stearate was disaggregated using a 40 mesh sieve and blended with the aforementioned blend for an additional 72 tumbles. Tooling, hardness and thickness for the various tablets were as follows:

| | | Hardness | Thickness |
|---|---|---|---|
| | Tooling | (kp) | (inches) |
| 40 mg (slow) | 0.343″ round standard concave | 7 | 0.166 |
| 40 mg (slow) | 0.2500 × 0.4650 arc-diamond | 10 | 0.195 |
| 40 mg (fast) | 0.2500 × 0.4650 arc-diamond | 9 | 0.117 |

| | Slow-Release | |
|---|---|---|
| | Formulation | |
| | Lot 01153-036 | Fast-Release Formulation |
| Material | (% w/w) | Lot 01153-050 (% w/w) |
| Oxycodone | 16.7 | 16.7 |
| Hydrochloride, | | |
| USP | | |
| Slow ProCR | 60.0 | NA |
| Fast ProCR | NA | 27.5 |
| Microcrystalline | 18.0 | 55.6 |
| Cellulose and | | |
| Colloidal Silicon | | |
| Dioxide | | |
| (ProSolv HD90) | | |
| Magnesium | 0.25 | 0.25 |
| Stearate | | |

Those skilled in the art will recognize, or be able to ascertain using no more than routine experimentation, many equivalents to specific embodiments of the invention described herein. Such equivalents are intended to be encompassed in the scope of the following claims.

We claim:

1. A matrix-forming, sustained-release pharmaceutical formulation comprising:
i) an effective amount of at least one drug substance;
ii) at least one water-swellable, pH independent polymer;
iii) at least one anionic, pH-dependent, gel-forming copolymer; and
iv) at least one polymer selected from the group consisting of:
a. a cationic polymer; and
b. a hydrocolloid.

2. A matrix-forming, sustained-release pharmaceutical formulation for oral administration comprising:
i) an effective amount of at least one drug substance;
ii) at least one water-swellable, pH independent polymer;
iii) at least one anionic, pH-dependent, gel-forming copolymer; and
iv) at least one polymer selected from the group consisting of
a. a cationic polymer; and
b. a hydrocolloid.

3. A matrix-forming, sustained-release pharmaceutical formulation comprising:
i) an effective amount of at least one drug substance;
ii) at least one water-swellable, pH independent polymer;
iii) at least one anionic, pH-dependent, gel-forming copolymer; and
iv) at least one polymer selected from the group consisting of
a. a cationic polymer; and
b. a hydrocolloid,
the formulation of which is substantially free of non-aqueous solvent.

4. A pharmaceutical formulation according to claim 2 wherein the in vitro release profile of at least one of said drug substance at intestinal pH is near linear.

5. A pharmaceutical formulation according to claim 2 wherein the in vitro release profile of at least one of said drug substance at gastric pH is substantially a first-order release profile.

6. A pharmaceutical formulation according to claim 2 wherein the in vitro release profile of at least one of said drug substance at gastric pH is near linear.

7. A pharmaceutical formulation according to claim 2 wherein the release of at least one of said drug substance is over a period of greater than about four hours.

8. A pharmaceutical formulation according to claim 2 wherein the release of at least one of said drug substance is over a period of greater than about eight hours.

9. A pharmaceutical formulation according to claim 2 wherein the release of at least one of said drug substance is over a period of greater than about twelve hours.

10. A pharmaceutical formulation according to claim 2 wherein the release of at least one of said drug substance is over a period of about twenty-four hours.

11. A pharmaceutical formulation according to claim 2 wherein the pharmacological effect from at least one of said drug substance lasts at least about four hours.

12. A pharmaceutical formulation according to claim 2 wherein the pharmacological effect from at least one of said drug substance lasts at least about eight hours.

13. A pharmaceutical formulation according to claim 2 wherein the pharmacological effect from at least one of said drug substance lasts at least about twelve hours.

14. A pharmaceutical formulation according to claim 2 wherein the pharmacological effect from at least one of said drug substance lasts at least about twenty-four hours.

15. A pharmaceutical formulation according to claim 2 wherein the aqueous solubility of the at least one drug substance is selected from the group consisting of high, moderate, low, low to moderate and moderate to high.

16. A pharmaceutical formulation according to claim 2 wherein the at least one drug substance is selected from the group consisting of hydrophobic and hydrophilic.

17. A pharmaceutical formulation according to claim 2 wherein the formulation is optionally coated, said coating comprising at least one pharmaceutically non-functional coating.

18. A pharmaceutical formulation according to claim 2 wherein the formulation is optionally coated, said coating comprising at least one pharmaceutically functional coating.

19. A pharmaceutical formulation according to claim 2 wherein the formulation is optionally coated, said coating further comprises one or more drug substances wherein said one or more drug substances is the same or different than the one or more drug substances contained in said matrix.

20. A pharmaceutical formulation according to claim 2 in tablet form.

21. A pharmaceutical formulation according to claim 2 in capsule form.

22. A pharmaceutical formulation according to claim 21 wherein said capsule is filled with a substance in the form selected from the group consisting from at least one mini-tab, multi-particulates and a plug.

23. A pharmaceutical formulation according to claim 2 when at least one step in preparing said formulation is prepared using direct compression.

24. A pharmaceutical formulation according to claim 2 when at least one step in preparing said formulation is prepared using dry granulation.

25. A pharmaceutical formulation according to claim 2 when at least one step in preparing said formulation is prepared using wet granulation.

26. A pharmaceutical formulation according to claim 2 having a bi-phasic release profile.

27. A pharmaceutical formulation according to claim 2 wherein said at least one drug substance is 1-methylnicotinamide or a pharmaceutically acceptable salt thereof.

28. A pharmaceutical formulation according to claim 27 wherein said salt is the hydrochloric salt.
